(19) United States
(12) Patent Application Publication
(10) Pub. No.: US 2025/0248621 A1
(43) Pub. Date: Aug. 7, 2025

(54) WEARABLE SENSOR-BASED DEVICE

(71) Applicant: University of Kentucky Research Foundation, Lexington, KY (US)

(72) Inventors: Madison Bates, Lexington, KY (US); Sridhar Sunderam, Lexington, KY (US)

(21) Appl. No.: 19/044,196

(22) Filed: Feb. 3, 2025

Related U.S. Application Data
(60) Provisional application No. 63/549,003, filed on Feb. 2, 2024.

(51) Int. Cl. A61B 5/11; A61B 5/00

(52) U.S. Cl. CPC A61B 5/1125; A61B 5/6826; A61B 5/7246; A61B 2505/09; A61B 2560/0238

(57) ABSTRACT

Certain aspects of the present disclosure provide techniques for generating a performance measure for a specified hand task. Such techniques may include receiving sensor data from a plurality of hand-mounted sensors configured to measure at least one of finger flexion or applied force by multiple digits of a hand during a performance of a specified task; processing the received sensor data to transform the sensor data into an output indicative of at least one of motion or force application patterns for the specified task; and generating a comparison measure based on the output and reference data associated with the same specified task, wherein the comparison measure indicates at least one of a level of similarity or level of discrepancy between a measured performance for the specified task and a baseline for the specified task.

### CROSS-REFERENCE TO RELATED APPLICATIONS

This Application claims the benefit of and priority to U.S. Provisional Patent Application No. 63/549,0003, filed on Feb. 2, 2024, the entire contents of which are hereby incorporated by reference.

### INTRODUCTION

### Field of the Disclosure

Aspects of the present disclosure relate to evaluating hand function and, more particularly, to techniques for capturing and analyzing sensor data corresponding to a user's hand function.

### Description of Related Art

Neurovascular diseases, particularly strokes, are a leading cause of physical and cognitive impairment worldwide. Strokes significantly impact hand function, with many stroke survivors experiencing reduced mobility. Additionally, a large portion of stroke survivors suffer from upper extremity impairments, which severely affect their ability to perform activities of daily living (ADLs). The rehabilitation process for stroke patients is often lengthy and requires continuous monitoring and assessment to track progress and adjust treatment plans accordingly.

Current clinical assessments for hand function in stroke patients are predominantly subjective and time-consuming. These assessments, such as the Fugl-Meyer Assessment (FMA), Action Research Arm Test (ARAT), and Box and Block Test, rely on the expertise of clinicians to evaluate the severity of impairments and the effectiveness of rehabilitation interventions. However, these assessments often use coarse scoring systems and lack the precision needed for detailed analysis of hand function. Moreover, the subjective nature of these assessments can lead to variability in results, making it challenging to obtain consistent and reliable data.

Advancements in wearable technology have opened new avenues for objective and quantitative assessment of hand function. Wearable devices have the potential to provide continuous monitoring and offer real-time feedback, which is important for accurately tracking the progress of stroke rehabilitation. These devices can capture detailed information about hand movements and the forces exerted during various tasks, providing a more comprehensive understanding of a patient's hand function.

Integrating wearable technology into clinical assessment protocols can significantly enhance the accuracy and efficiency of hand function evaluations. These devices can validate clinical assessment scores, correlate data with traditional assessment metrics, and facilitate the development of personalized rehabilitation programs. This approach can potentially improve the overall outcomes of stroke rehabilitation and enhance the quality of life for stroke survivors.

### SUMMARY

One aspect provides a method for generating a performance measure for a specified hand task. Such methods may include receiving sensor data from a plurality of hand-mounted sensors configured to measure at least one of finger flexion or applied force by multiple digits of a hand during performance of a specified task; processing the received sensor data to transform the sensor data into an output indicative of at least one of motion or force application patterns for the specified task; and generating a comparison measure based on the output and reference data associated with the same specified task, wherein the comparison measure indicates at least one of a level of similarity or level of discrepancy between a measured performance for the specified task and a baseline for the specified task.

Other aspects provide: an apparatus operable, configured, or otherwise adapted to perform any one or more of the aforementioned methods and/or those described elsewhere herein; a non-transitory, computer-readable media comprising instructions that, when executed by a processor of an apparatus, cause the apparatus to perform the aforementioned methods as well as those described elsewhere herein; a computer program product embodied on a computer-readable storage medium comprising code for performing the aforementioned methods as well as those described elsewhere herein; and/or an apparatus comprising means for performing the aforementioned methods as well as those described elsewhere herein. By way of example, an apparatus may comprise a processing system, a device with a processing system, or processing systems cooperating over one or more networks.

The following description and the appended figures set forth certain features for purposes of illustration.

### BRIEF DESCRIPTION OF DRAWINGS

The appended figures depict certain features of the various aspects described herein and are not to be considered limiting of the scope of this disclosure.

FIG. 1 depicts an exemplary overview of a system that measures and analyzes user hand movements in real-time, in accordance with some aspects of the present disclosure.

FIGS. 2A-2D depict various embodiments of sensor arrangements on a user's hand in accordance with some aspects of the present disclosure.

FIG. 3 depicts a set of illustrative tasks or movements in accordance with some aspects of the present disclosure.

FIGS. 4A-4B depict example graphical user interfaces and flow diagrams for planning, performing, and resting between hand function tasks, in accordance with some aspects of the present disclosure.

FIG. 5 depicts a metric processor architecture in accordance with some aspects of the present disclosure.

FIGS. 6A-6D depict return map trajectories obtained from multiple participants performing different object manipulation tasks, in accordance with some aspects of the present disclosure.

FIGS. 7A-7B depict radar-style charts illustrating flex and force distributions for various grasping or manipulation tasks in accordance with some aspects of the present disclosure.

FIG. 8 depicts a comparison between original and time-aligned signals using dynamic time warping in accordance with some aspects of the present disclosure.

FIGS. 9A-9D depict pairwise distance matrices derived from dynamic time warping across multiple tasks and participants in accordance with some aspects of the present disclosure.

FIG. 10 depicts an exemplary comparison component in accordance with some aspects of the present disclosure.

FIG. 11 depicts a high-level illustration of brain signal acquisition, feature extraction, and proprioceptive feedback delivered via a sensor-based and/or assistive device in accordance with some aspects of the present disclosure.

FIG. 12 depicts a method for generating a performance measure for a specified hand task in accordance with some aspects of the present disclosure.

FIG. 13 depicts aspects of an example processing system.

### DETAILED DESCRIPTION

Aspects of the present disclosure provide apparatuses, methods, processing systems, and computer-readable media for capturing sensor data corresponding to a user's hand function, transforming that data with signal analysis techniques, and generating feedback or comparison measures indicative of the user's performance during specific tasks. More particularly, some aspects described herein leverage time-alignment methods (e.g., dynamic time warping), dimensionality reduction (e.g., principal component analysis), and other data transformations (e.g., delay embedding) to model and visualize the dynamics of hand movements, compare them against nominal references, and produce metrics for guiding rehabilitation or diagnostic interventions.

A technical problem arises in objectively evaluating and tracking a user's (e.g., a patient's) hand function over time, especially when the user performs routine yet variable tasks like picking up objects, manipulating handles, or applying force. Traditional assessments often rely on coarse, subjective rating scales or time-consuming manual evaluations that may not capture subtle improvements or deficits. Moreover, sensor data from multiple fingers and force channels tend to be high-dimensional, noisy, and subject to variable speeds or incomplete cycles, making direct comparisons between users or sessions difficult. Existing approaches struggle to account for these irregularities, leading to less reliable clinical decisions and slower adjustment of therapy plans.

To address these technical challenges, certain aspects are directed at collecting sensor data from a user's hand via force or flex sensors and then transforming the collected sensor data using one or more processing techniques. In some aspects, a processing system can filter and align the sensor data, reducing extraneous noise and adjusting for speed or phase disparities via dynamic time warping or similar algorithms. Principal component analysis (PCA) or similar algorithms can reduce dimensionality to capture dominant movement synergies, while delay embedding or other phase-space representations can reveal underlying dynamics of movement patterns. The transformed data can then be compared against nominal or reference metrics (e.g., from different user baselines or the same user's historical data) to generate a quantitative measure of similarity or discrepancy. In some aspects, the system may enable more objective, fine-grained assessments of hand function, which can be used to tailor or update treatment protocols.

In some aspects, techniques described herein provide several technical advantages. First, such techniques may handle temporal misalignment in sensor signals, addressing speed or pause variations. Second, such techniques may reduce the dimensional complexity of multi-finger flex and force data by focusing on clinically meaningful motor synergies. Third, by generating an output representation (e.g., a return map or PCA plot) and a comparison measure, the aspects described herein can provide intuitive, data-driven insights into a patient's progress. Such real-time or near real-time feedback can provide for refinements to a therapy or diagnostic plan. Accordingly, such a system may provide for scalable and customizable deployments, accommodating diverse tasks (e.g., twisting lids, grasping objects, opening drawers) while performing evaluations even in noisy, real-world rehabilitation environments.

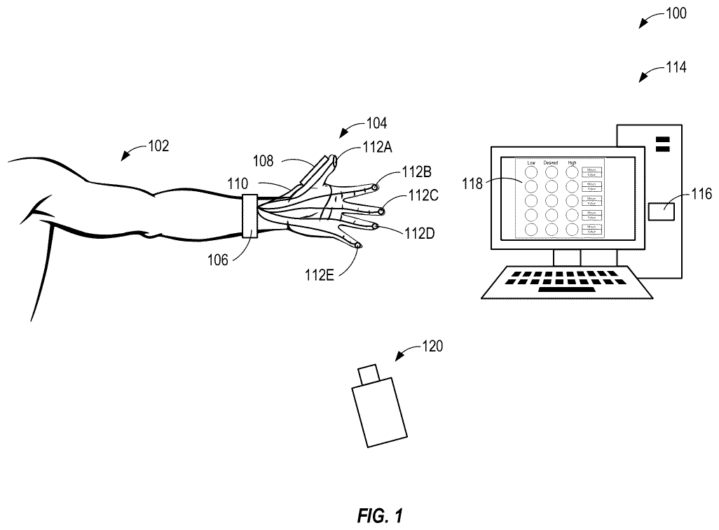

Referring to FIG. 1, an exemplary system 100 is depicted, which may be configured to measure and analyze various aspects of hand function during user interactions. In certain aspects, system 100 may include one or more sensors configured to detect flexion, force application, or other parameters relevant to a user's hand and arm movement. In some aspects, these measurements may be relayed to a processing system for data analysis, visualization, or further processing. While FIG. 1 depicts one exemplary configuration, those skilled in the art will appreciate that various modifications to layout, communication protocols, and sensor types may be made without departing from the scope and spirit of the invention.

In some aspects, an arm 102 of a user may be any portion of the upper extremity extending from the user's shoulder down to the wrist, thereby providing a foundation for positioning sensors that measure physiological or biomechanical activity. In some aspects, arm 102 is shown wearing a wrist-mounted sensor communication board 106, which may be attached using a strap, cuff, or other securing mechanism suitable for maintaining the stable alignment of one or more sensors. The user's arm 102 may be of various dimensions and shapes, and the system 100 may be configured to accommodate users with different anatomical proportions.

In some aspects, arm 102 may work as the main support structure for a sensor array, ensuring that readings from the hand 104 can be stably captured. In certain implementations, padding or protective material may be included around arm 102 to increase user comfort and facilitate long-term wear during rehabilitation exercises or repeated data collection sessions. Additionally, the geometry of arm 102 may be measured or calibrated at the outset of using the system 100 to ensure that sensor placement is precise and that the sensor readings correlate accurately to relevant biomechanical or physiological parameters.

Moreover, arm 102 may move through multiple planes of motion, such as flexion, extension, supination, and pronation, and the system 100 can be configured to track or correlate these arm movements with hand movements performed by hand 104. By referencing the position and orientation of arm 102, the system 100 can derive additional contextual information. In some aspects, the additional contextual information may include the user's overall posture and the orientation of the hand 104 relative to the rest of the body.

In some aspects, the hand 104 is the focal point of a sensing arrangement, as the hand performs various movements monitored by the system 100. In some aspects, such movements may include, but are not limited to opening, closing, gripping, pinching, and applying pressure to objects. In some aspects, the hand 104 may wear individual finger sensors, such as force-sensitive resistors 112A-112E and one or more flex sensors 108 positioned along the back of the hand and/or along specific fingers.

The hand 104 may have a complex range of motion, including flexion and extension at the metacarpophalangeal joints, abduction and adduction of digits, and more subtle fine-motor movements. In some aspects, the system 100 may capture these motions and provide quantitative feedback, including but not limited to a user's dexterity, grip strength, and coordination levels. For example, in rehabilitation settings, the data collected from the hand 104 may be used to track a patient's progress, identify specific impairments, or customize therapy protocols.

In certain aspects, hand 104 can be oriented in various ways relative to arm 102, and system 100 can incorporate or integrate additional reference markers to track this orientation. Further, the system 100 can adapt to different hand sizes and shapes using adjustable finger sleeves or sensor modules. As the user's comfort may be important, typically wearable components around hand 104 may be composed of flexible, breathable materials, allowing prolonged or repeated usage without discomfort. Additionally, an interface layer, such as a thin glove-like material and/or flexible 3D printed material, may separate the force sensors 112A-112E from direct contact with the skin, thereby protecting both the sensors and the user's hand. Alternatively, or in addition, such sensors may be encapsulated within a glove-like material, whereby such sensors may or may not make direct contact with a user's skin or an external object, such as a tennis ball, coffee cup, lid, or handle.

Moreover, hand 104 may be involved in various validation or calibration tasks involving an optical device 120, as discussed below. During such validation sessions, the user's hand might repeatedly perform a known range of motions so that system 100 can correlate sensor readings with ground-truth measurements or known motion patterns. Accordingly, the data collected from hand 104 may be consistent and accurate over time, even if external conditions or sensor orientations change.

In some aspects, a sensor communication board 106, which may include one or more data acquisition modules, power units, or wireless communication interfaces, may be affixed around the user's wrist or forearm, ensuring that such sensor communication board 106 remains securely in place during operation. The sensor communication board 106 may be configured to receive analog or digital signals from sensors (such as flex sensor 108 and force sensors 112A-112E) via sensor wires 110, and the sensor communication board 106 may convert these signals into a suitable format for transmission to the processing system 114.

In certain implementations, sensor communication board 106 includes an onboard processor or microcontroller capable of handling preliminary signal conditioning, such as but not limited to filtering noise, performing analog-to-digital conversion, or executing sensor-specific calibration routines. The sensor communication board 106 may also regulate power distribution to the various sensors (e.g., flex sensors 108 and/or FSR sensors 112), ensuring that each sensor receives an appropriate voltage and that the system conserves battery life where applicable. In some aspects, the sensor communication board 106 may incorporate multiplexing capabilities so that multiple sensor channels can share communication lines or reduce overall wiring complexity.

Moreover, the sensor communication board 106 may incorporate wireless modules (e.g., Bluetooth® Low Energy, Wi-Fi®, or proprietary RF technology) to enable real-time (or near real-time) data streaming to the external signal processing component 116 or the broader processing system 114. This wireless functionality may be advantageous in situations where the user needs freedom of movement, such as during rehabilitation exercises, tasks of daily living, or clinical assessments requiring minimal tethering to external hardware.

In addition, the sensor communication board 106 may house memory storage to log sensor data for offline analysis. In some aspects, the logged data can be used to analyze the user's movements or provide a backup in the event of a wireless transmission failure. Should the user prefer a wired setup, the sensor communication board 106 can also include ports for USB or other serial connections, facilitating direct data output to the processing system 114. Accordingly, the sensor communication board 106 may be a central hub for data acquisition, communication, and preliminary processing, bridging the physical sensors (e.g., 108, 112A-112E) with the signal processing and user interface components of system 100.

In some aspects, the flex sensor 108 is positioned along or near one or more fingers of hand 104 or the dorsal side of the hand. In some aspects, the flex sensor 108 may be configured to detect bending or changes in curvature, thereby translating mechanical deformation into a corresponding electrical signal. For instance, as a user bends the knuckles or extends the fingers, flex sensor 108 may produce measurable variations in resistance or voltage, reflecting the flexion degree.

In certain aspects, flex sensor 108 comprises a thin, flexible substrate with conductive or resistive elements that elongate or compress when bent. The flex sensor 108 may be calibrated to detect a wide range of finger angles, from minimal flexion (near full extension) to maximum contraction (a closed fist). By correlating the signal from flex sensor 108 with reference data acquired from an optical device 120 or other calibration devices, system 100 can generate accurate angle estimates for each measured finger joint.

Moreover, flex sensor 108 may communicate with sensor communication board 106 through sensor wire 110. This wired connection may carry low-voltage signals representing the instantaneous flexion state of the user's hand 104. The data sampling frequency can be adjusted as desired; for instance, a higher sampling rate might be used during rapid finger movements, while a lower rate might suffice for more static or isometric tasks. Because the flex sensor 108 is small and light, the flex sensor 108 may minimally interfere with the user's natural hand movements, which may be particularly advantageous in therapeutic or clinical contexts, where preserving a natural range of motion is important when under assessment. In certain implementations, flex sensor 108 may be integrated into a glove-like garment that the user dons and the sensor's contact points may be reliably positioned about the hand 104. Alternatively, flex sensor 108 might be affixed using removable adhesives or straps directly onto the skin or external supports. Accordingly, the flex sensor 108 facilitates the capture of dynamic movement data correlated with force data from sensors 112A-112E to provide a more complete perspective of the user's hand movement.

In some aspects, the sensor wire 110 may refer to a sensor wire that electrically and/or communicatively couples flex sensor 108 (and possibly other sensors) to sensor communication board 106. In certain aspects, the sensor wire 110 includes multiple conductive strands or channels to accommodate both data signals and power lines, ensuring that the sensor is integrated into a data acquisition system, such as that provided by the sensor communication board 106.

In some aspects, the sensor wire 110 can be routed along arm 102 to avoid tangling or interference with the user's movement. The wire's insulation may be chosen for flexibility, durability, and user comfort, as well as for protection against electromagnetic interference in clinical or laboratory environments. In some cases, sensor wire 110 may feature shielding to reduce noise from external sources, promoting higher fidelity in the captured signals.

In operation, sensor wire 110 may transmit low-voltage analog or digital signals to sensor communication board 106, where initial signal processing may occur before the data is forwarded to processing system 114. Because sensor wire 110 can be subject to repetitive bending during normal usage, it is often constructed from materials and designed to withstand mechanical fatigue over many cycles of flexion and extension. In some aspects, the sensor wire 110 can function as part of a sensor identification scheme, such that each sensor connected to the sensor communication board 106 can be recognized automatically based on pin configuration or wire color coding. Accordingly, setup can be simplified, and user error can be reduced when sensors are attached or reattached to system 100.

In some aspects, a finger-specific force-sensitive resistor (FSR) 112A-112E may be positioned on or near a respective fingertip to detect pressure or force the digit applies during a grasp, pinch, press, or other manipulation. For instance, FSR sensor 112A may be positioned on the thumb, FSR sensor 112B on the index finger, and so on, ensuring coverage of each finger that participates in typical hand function. FSRs 112A-112E generally operate by exhibiting a change in electrical characteristics when a force is applied. In some aspects, an output voltage signal may increase with applied force (e.g., higher force may generate a higher voltage output and/or a lower force may generate a lower voltage output). These voltage variations can be mapped to absolute or relative pressure or grip strength values. In certain aspects, the FSRs 112A-112E may be integrated into housings or embedded in flexible materials that conform to the user's fingertips, maintaining consistent contact. While the underlying mechanisms may involve changes in electrical resistance, the voltage output can provide a direct measurement that can be readily processed by the sensor communication board 106.

In some aspects, each FSR 112A-112E may be connected to sensor communication board 106 through wired connections (not specifically numbered in FIG. 1 but generally analogous to sensor wire 110), and signals from these sensors can be sampled concurrently or at staggered intervals. By collecting data from all five FSRs 112A-112E, system 100 can build a force distribution profile across the user's fingers, which may provide further insight during clinical assessments based on grip patterns, rehabilitative exercises, or objective measurements of dexterity.

In some configurations, the FSRs 112A-112E may be calibrated against known loads, ensuring measurement reliability. This calibration may be conducted using standardized weights or a load cell, and the calibration factors may be stored within the sensor communication board 106 or in the processing system 114. The sensors 112A-112E may also incorporate or overlay protective layers to safeguard the sensing elements from moisture, oils, or other contaminants that might affect performance. Moreover, by capturing fingertip forces in real-time, system 100 can provide immediate biofeedback to the user via a GUI 118, indicating whether they are applying the correct amount of pressure or distributing force evenly among their digits.

In some aspects, the processing system 114 may receive, store, analyze, and/or display sensor data gathered by the sensor communication board 106. In certain implementations, processing system 114 may be a desktop computer, laptop, tablet, or embedded device equipped with suitable software to handle data streams from flex sensor 108, FSRs 112A-112E, or any additional sensors. In some aspects, the processing system 114 may carry out various tasks, including filtering raw signals, computing kinematic parameters, rendering real-time visual feedback, or comparing measured movements against stored profiles of normative data. For example, the processing system 114 may compare a user's finger flexion angles with standard healthy ranges, or it might correlate fingertip force data with expected values based on the user's age, rehabilitation stage, or clinical diagnosis.

Additionally, processing system 114 may store historical data sets in a database, facilitating progress tracking over multiple sessions. Clinicians or researchers might access these data sets to evaluate hand function improvement or design personalized exercise regimens. In some aspects, processing system 114 can output data in standardized formats for further analysis in third-party software or integration with hospital electronic medical record systems. Moreover, the software running on processing system 114 can be customized to display advanced metrics, such as synergy patterns between fingers or dynamic force changes during complex tasks. In certain implementations, the processing system 114 may harness machine learning algorithms to detect subtle movement patterns, thereby offering predictive analytics about the user's recovery trajectory or the likelihood of performing certain tasks successfully.

In some aspects, the signal processing component 116 may be embodied as a discrete hardware module within the processing system 114, as a specialized software routine, or as a combination of hardware and software elements. In some aspects, the signal processing component 116 may clean, condition, and transform raw sensor signals into meaningful parameters. For example, the signal processing component 116 may apply noise-reduction algorithms to remove artifact signals from the flex sensor 108 or FSRs 112A-112E. This could involve band-pass filters, wavelet transforms, or other digital signal processing (DSP) techniques. Once the signals are cleaned, the signal processing component 116 can extract features such as peak force, rate of force development, angle increments, or duration of sustained grip. These features may then be passed on to higher-level analytical or application layers within the processing system 114, where they can provide immediate biofeedback via the GUI 118, for example. In rehabilitation contexts, signal processing component 116 may trigger alerts if the user's performance deviates from the expected level. For instance, if the user suddenly loses grip force while attempting to hold an object, the signal processing component may trigger an alert. Moreover, the signal processing component 116 may facilitate real-time control loops. For example, suppose a user is participating in an interactive training program. In that case, the processed signals from the signal processing component 116 can be used to dynamically adjust difficulty levels in a virtual environment or to control external devices, such as rehabilitation robots or functional electrical stimulation units.

In some embodiments, the signal processing component 116 is implemented on a field-programmable gate array (FPGA) or a microcontroller for low-latency operation. Alternatively, the signal processing component 116 could be executed as part of a software library on a general-purpose CPU. The architecture choice may depend on whether system 100 is to have portable, low-power solutions (for home use) or robust, high-throughput solutions (for advanced clinical or research settings). In either case, the refined data output of signal processing component 116 may provide accurate, real-time analysis of the user's hand function.

In some aspects, the example graphical user interface (GUI) 118 may be displayed on a monitor or screen in communication with the processing system 114. The GUI 118 may be configured to convey sensor readings, feedback, and interactive prompts to the user or clinician in real-time or near real-time. For instance, the GUI 118 may display visual indicators representing the degree of finger flexion, the amount of force being applied by each finger, or composite metrics such as grip strength or pinch accuracy.

In rehabilitation, the GUI 118 can guide the user through exercises by showing targets or thresholds for force or movement. As the user moves their fingers or applies pressure, the GUI 118 can immediately reflect whether the user is below, within, or above the recommended range. Such feedback mechanisms help keep users engaged, providing a sense of accomplishment when they meet prescribed goals. Additionally, the GUI 118 can log the user's performance over multiple sessions, offering progress charts or heat maps that pinpoint which fingers exhibit improvements in strength or precision. Clinicians might use these visual summaries to update therapy strategies or to highlight areas requiring further attention. The GUI 118 can be customized for different types of tasks. For example, a more straightforward, large-text interface for geriatric patients or more advanced graphics and gamified challenges for younger users may be displayed by the GUI 118.

In some aspects, an optical device 120 may be employed to validate the sensor readings obtained by flex sensor 108. In some aspects, an optical device 120, such as a camera, may include a motion capture device, such as but not limited to a Leap Motion Controller (LMC), capable of optically detecting and tracking the position and orientation of the user's hand and fingers in three-dimensional space. By capturing real-time 3D hand data, the optical device 120 serves can provide an external reference or “ground truth” measurement. One of the primary reasons for including optical device 120 in system 100 is to ensure that the internal sensor readings (i.e., flex angles or applied forces) correlate accurately with actual physical movements. In many applications, particularly those involving rehabilitation or clinical diagnostics, a high degree of accuracy and repeatability is needed. To that end, the optical device 120 can detect subtle changes in finger location or orientation, and these measured changes can be compared to the corresponding signals from flex sensor 108 or each force-sensitive resistor 112A-112E. Since optical tracking generally requires direct line of sight to function properly, the optical device 120 may be primarily used for sensor validation rather than during clinical assessments where hand movements might obstruct the camera's view.

For example, during a validation protocol, the user may be asked to perform a series of standardized motions—such as repeated pinching, full hand extension, or gripping an object. As these motions are executed, the optical device 120 records the three-dimensional trajectory of each fingertip. Meanwhile, system 100's sensor communication board 106 and processing system 114 record data from the flex sensors 108. The data sets may then be aligned, and the correlations between the optical readings and the sensor signals are computed. A strong correlation or minimal error margin indicates that the system's 100 sensors function correctly, while any discrepancies might prompt recalibration or sensor replacement. This validation step may bolster the reliability of system 100, ensuring that the system's 100 output, such as feedback to a real-time GUI 118 or computed metrics, accurately reflect actual user movements. Such accuracy may be important in medical or rehabilitative environments, where decisions regarding a patient's progress and subsequent treatments rely on precise measurements.

In addition to calibration, the optical device 120 can provide ongoing quality control needs. For instance, if, over time, the flex sensor 108 drifts due to wear or environmental factors, comparison against the feed of the optical device 120 can detect anomalies early. Thus, optical device 120 not only establishes confidence in the system's baseline performance but also maintains consistent measurement fidelity throughout the life cycle of system 100.

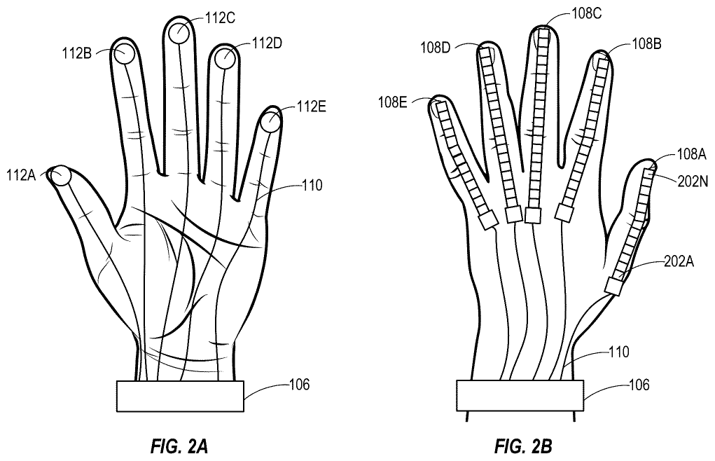

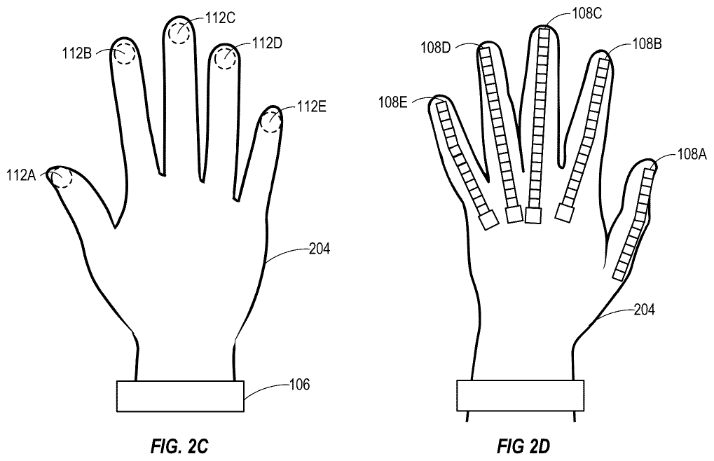

Referring now to FIG. 2A, an example force-sensing arrangement for individual fingers in a hand-based sensing system is depicted in accordance with aspects of the present disclosure. As illustrated in FIG. 2A, one or more multiple finger-specific FSRs 112A-112E are disposed at locations corresponding to a user's thumb, index finger, middle finger, ring finger, and pinky. In some aspects, these sensors are operatively connected to a sensor communication board 106 through one or more sensor wires 110. In some aspects, a wireless communication pathway may be utilized in addition to, or in lieu of, physical wiring, thereby transmitting sensor data to the sensor communication board 106 and/or a separate processing system (e.g., 114 in FIG. 1). In some aspects, the sensors and wiring may be folded over the dorsal (e.g., back) side of the hand; such an approach can keep the palm unobstructed and enable more natural interaction with objects during activities of daily living.

As previously described, the FSR sensor 112A may be aligned with a user's thumb, as shown in FIG. 2A, the FSR sensor 112A is depicted near the distal portion of the thumb, where it is positioned to detect pressure or force exerted during gripping, pressing, or pinching motions. In certain implementations, FSR sensor 112A may be adhered to a glove-like substrate or attached directly to the skin via a medical-grade adhesive. By situating FSR sensor 112A at the thumb, the system can capture data related to thumb-specific activities, such as picking up small objects or interacting with touchscreens.

Because the thumb is central to many dexterous tasks, FSR sensor 112A often experiences the greatest variation in force range during day-to-day use. Consequently, 112A may be calibrated or manufactured with a wider dynamic range than the other sensors, ensuring it can measure everything from gentle tapping forces to more forceful presses. This calibration process can involve applying known force values to 112A while capturing the corresponding electrical output. Once properly calibrated, FSR sensor 112A's readings can be provided to the sensor communication board 106, where, in some aspects, initial signal conditioning or filtering may occur. The sensor communication board 106 may communicate the data to a higher-level processor for real-time visual feedback or offline analysis, enabling healthcare professionals or users to track improvements in thumb strength or dexterity over time.

FSR sensor 112B is the force-sensitive resistor positioned at the user's index finger, typically near or at the fingertip. The index finger is heavily utilized in tasks requiring precision, such as typing, pressing small switches, or pointing. By placing FSR sensor 112B at the distal portion of the index finger, the system can capture subtle variations in force that occur during fine motor activities. For instance, during tasks like buttoning a shirt or threading a needle, FSR sensor 112B's measurements can reveal micro-adjustments in applied force that might not be detectable by less sensitive methods.

In various embodiments, the physical design of FSR sensor 112B may involve a thin, flexible sensor material that readily conforms to the fingertip's shape without significantly impeding natural movement. In some aspects, the FSR may be polymer-based, while in other aspects, a piezoresistive layer may be integrated into a laminate structure. Once connected to the sensor communication board 106 (either by sensor wire 110 or wirelessly), FSR sensor 112B's signal can be sampled at a rate sufficient to track rapid fluctuations in force. This sampling rate can be adjusted in real time based on the application's needs. For example, a sample rate may be configured to a higher rate for tasks involving quick tapping motions. Accordingly, the system can identify patterns in FSR sensor 112B's force data, including consistent under-or over-application of force, which may indicate potential motor impairments and/or training opportunities.

FSR sensor 112C represents the force-sensitive resistor situated at the middle finger. The middle finger often contributes significant force during tasks that involve a strong grip, such as grasping tools, lifting heavier objects, or providing stability to the hand during precision grips. As illustrated in FIG. 2A, FSR 12C may be placed at or near the fingertip to register direct contact forces, though alternative embodiments can locate it along the finger's phalanges or near the joint area to capture a broader profile of pressure distribution.

In many configurations, FSR 112C's placement is important for capturing a range of forces that vary from light contact (e.g., gentle tapping) to more robust grip pressures (e.g., clenching a ball). Depending on the user's rehabilitation needs or performance goals, FSR sensor 112C may be combined with other sensors (e.g., additional motion or angle trackers) to correlate force data with finger flexion. The signals originating from FSR sensor 112C may be provided to the sensor communication board 106 via sensor wire 110 or wirelessly. Additionally, FSR sensor 112C may be fitted with protective overlays to shield the sensing element from abrasion or moisture, ensuring durability and reliability over extended periods of repetitive use.

FSR sensor 112D is a force-sensitive resistor that interfaces with the ring finger. This finger often plays a supportive or stabilizing role during object manipulation, working in conjunction with the other fingers to distribute grip forces more evenly. In many day-to-day tasks, the ring finger's force contribution is somewhat less than the index or middle finger, but it still provides important feedback about the overall grip strategy. By monitoring FSR sensor 112D, the system can detect whether the user is compensating with other digits or whether they are applying consistent force across all fingers when lifting or holding objects.

The physical construction of FSR sensor 112D may mirror that of FSR sensor 112B or FSR sensor 112C, involving a laminated sensor stack with a pressure-sensitive layer. During usage, FSR sensor 112D's electrical resistance may change as pressure is applied, which may then be read and converted into a force estimate by the sensor communication board 106 or a subsequent signal processing module. In certain aspects, the FSR sensor 112D may incorporate a slightly different sensitivity threshold compared to index or middle finger sensors, reflecting typical loads encountered by that digit. For example, if FSR sensor 112D is primarily used in tasks involving the placement of smaller, finer forces, the dynamic range might be optimized for lower values, whereas high-force applications might necessitate a different sensor specification and, therefore, a different dynamic range. Regardless of design choices, FSR sensor 112D's data can be combined with the other four FSR signals from FSR sensors 112A, 112B, 112C, and 112E to create a more complete snapshot of the user's grip profile.

FSR sensor 112E is a force-sensitive resistor aligned with the user's pinky finger. Despite often being considered the least forceful digit during a full-hand grip, the pinky is integral to fine motor tasks and contributes to grip stability, especially in tasks requiring hand enclosure around an object (e.g., holding a cylindrical handle). Placing FSR sensor 112E on or near the pinky's fingertip provides the system with an additional dimension of force distribution data.

In some aspects, FSR sensor 112E can detect micro-forces, such as gentle balancing or object cradling, thereby yielding insights into the user's or patient's level of motor control in that digit. During routine tasks, the pinky may show less force variation compared to the thumb or index finger; however, the system can calibrate FSR sensor 112E to accurately register small or moderate pressures. This calibration may be dynamic, adjusting the sensor's sensitivity based on real-time comparisons of data from the other four FSRs (e.g., FSR 112A-112D). Data from the FSRs 112A-112E may be merged at sensor communication board 106 for additional analysis. Such analysis can indicate finger fatigue, identify post-stroke impairments, or track improvements in dexterity over repeated therapy sessions.

As previously described, the sensor communication board 106 may act as the data acquisition hub for the FSR array 112A-112E. As shown in FIG. 2A, the sensor communication board 106 is positioned near the user's wrist or palm area (though it can be located elsewhere, such as on the forearm, depending on design preferences). The sensor communication board 106 can aggregate signals from multiple sensors, converting raw analog data into digital form and optionally performing preliminary processing. For instance, sensor communication board 106 can handle tasks such as offset compensation, filtering, or detection of signal spikes that could indicate abrupt force changes.

In certain aspects, the sensor communication board 106 provides wired connectivity, via a separate or shared sensor wire 110, to each FSR (112A-112E). However, the sensor communication board 106 can also incorporate one or more wireless modules (e.g., Bluetooth®, Wi-Fi®, or proprietary radio) to communicate sensor readings to an external processing system (e.g., processing system 114 of FIG. 1). This wireless capability is beneficial when the user needs to perform tasks with minimal mechanical hindrance. Additionally, the sensor communication board 106 can store calibration parameters for each sensor, enabling consistent force measurements across multiple sessions.

As previously described, it should be understood that each of the force sensitive resistors 112A-112E may be coupled to sensor communication board 106 via its own dedicated sensor wire 110. In some implementations, each sensor wire 110 can be routed discretely along a glove or harness structure, preventing tangling or electrical interference. Alternatively, a single multi-conductor cable might bundle sensor wires from all five FSRs 112A-112E into one harness that terminates at sensor communication board 106. In certain applications, the FSRs 112A-112E may incorporate wireless transmitters themselves, making sensor wires 110 unnecessary for data transfer. Instead, each sensor could communicate directly with sensor communication board 106 or a remote processing system, thus further reducing mechanical constraints on the user's hand.

In various aspects, the FSR is a type of sensor that changes its electrical resistance in response to an applied force or pressure. At a high level, an FSR typically comprises a semiconductive layer that exhibits variable conductivity when pressed, sandwiched between two conductive substrates. When no force is applied, the semiconductive material maintains a relatively high electrical resistance. As incremental force is exerted, microscopic contact points within the semiconductive layer increase, allowing electric current to pass more readily. This results in a measurable decrease in electrical resistance, which can be translated into an approximate force reading.

Many FSR configurations rely on printed interdigitated electrodes on a flexible film. Once layered with a pressure-sensitive material, the entire assembly can be encapsulated for durability. A typical readout circuit involves measuring the voltage across the sensor using a reference resistor and forming a voltage divider. Changes in the FSR's resistance cause the output voltage to shift accordingly. The dynamic range and sensitivity of an FSR can be tuned by altering the thickness or composition of the semiconductive layer, the geometry of the electrodes, or the reference resistor value.

Because FSRs are generally low-cost, lightweight, and thin, they are widely used for applications such as wearable electronics, robotics, and interactive devices. However, FSRs are not typically designed for highly linear or absolute force measurements over large ranges. Instead, they provide repeatable trends that allow for relative force sensing within a specific application window. Calibration is often performed to map the sensor's raw output to approximate force units. Factors like temperature, humidity, and repeated bending can affect an FSR's baseline resistance. Thus, configurations that employ FSRs (like sensors 112A-112E) often include compensation strategies—such as real-time recalibration or filtering—to maintain measurement consistency.

Turning now to FIG. 2B, an exemplary configuration of a flex-sensing arrangement is depicted, illustrating how individual flex sensors may be configured along the length of each finger. In particular, FIG. 2B shows five finger-specific flex sensors 108A through 108E distributed to measure bending motions of the thumb, index finger, middle finger, ring finger, and pinky, respectively. Each flex sensor may be operably coupled to sensor communication board 106, and, as with the force sensors of FIG. 2A, data connection can be established using sensor wires 110 or through wireless communication means. Additionally, portions of each flex sensor are labeled as 202, indicating that multiple sensor elements or segments may be used to capture flex at various finger joints.

A flex sensor 108A may be positioned along the thumb. This flex sensor 108 can be implemented as a thin, elongated strip that changes its electrical characteristics based on the degree to which it is bent. Because the thumb exhibits a unique range of motion, flex sensor 108A can be structured or calibrated differently than the other flex sensors to capture the specific angles involved in thumb flexion, extension, or opposition. In certain aspects, flex sensor 108A may be attached with an adhesive or integrated into a fabric-based glove that conforms closely to the thumb's shape.

During operation, flex sensor 108A′s electrical output may be sampled by sensor communication board 106 at a configurable frequency, enabling real-time monitoring of thumb position. If the system employs a wireless communication strategy, flex sensor 108A can relay data via a transmitter embedded within or near the flex sensor 108A. Conversely, a sensor wire 110 may provide a reliable path to the sensor communication board 106. Regardless of the transmission mode, the raw signals from flex sensor 108A can be processed to infer the angular displacement of the thumb. This data may be useful during tasks like manipulating objects, using hand tools, or performing rehab exercises where thumb mobility is an important metric. By integrating flex sensor 108A with the rest of the finger flex sensors, the system can generate a complete profile of hand movement patterns and identify areas for improvement or targeted training in clinical contexts.

Flex sensor 108B corresponds to the flex sensor tailored to the index finger. Positioned typically along the dorsal or lateral aspect of the index finger, flex sensor 108B may measure how much the finger bends or extends during tasks such as tapping, pressing, or precision pointing. In some aspects, the flex sensor 108B may be segmented (as indicated by 202, discussed below) so that multiple points along the index finger can be measured, thus capturing nuances in the finger's joint movements. In other aspects, the flex sensor 108B may not require such segmentation to obtain measurements associated with the index finger.

Because the index finger frequently performs fine-grained tasks (e.g., pressing small keys or picking up tiny objects), flex sensor 108B may be calibrated for high sensitivity around the range of motion expected for typical index finger flexion. The flex sensor 108B may be attached using flexible adhesives or integrated directly into a wearable substrate, such as a glove. Signals from the flex sensor 108B may feed into sensor communication board 106 for immediate analysis or buffering, enabling real-time visualization of finger posture through a connected interface or machine-learning algorithms. In some rehabilitation scenarios, clinicians might rely on flex sensor 108B's readings to gauge incremental improvements in index finger dexterity, thus tracking a patient's progress over multiple therapy sessions.

Flex sensor 108C indicates a flex sensor deployed on the middle finger. This finger often engages in tasks requiring moderate to strong grip force, although precise control is also necessary in many day-to-day interactions. By placing a flex sensor along the middle finger, the system captures angular movement data that can be correlated with concurrent force measurements (if used in conjunction with force sensors) or with the user's overall grip pattern.

In some aspects, the flex sensor 108C may span from near the middle finger's proximal phalanx to the distal region, ensuring coverage of the primary joints. The flex sensor 108C may be constructed from durable yet flexible components that maintain consistent electrical characteristics over multiple cycles of use. When integrated with sensor communication board 106, flex sensor 108C's signals can be averaged, filtered, or aggregated with readings from other digits to form a more complete representation of the user's hand posture. In some aspects, the data from flex sensor 108C can be harnessed to assess coordination levels, detect anomalies in movement (e.g., tremors or partial range-of-motion deficits), and facilitate user feedback in the form of visual or haptic cues.

Flex sensor 108D refers to the flex sensor attached to the ring finger. The ring finger can play a significant role in maintaining grip strength, even though it is less individually dexterous than the index or middle fingers. By capturing real-time flexion data from flex sensor 108D, the system can identify whether the ring finger is synchronously moving with the other fingers or exhibiting delayed or restricted motion.

In some implementations, flex sensor 108D includes multiple sensing regions (see 202 discussion) that track proximal and distal joint angles independently, yielding more granular insights into ring finger motion. The sensor's output, typically expressed as a change in resistance or capacitance, is relayed to sensor communication board 106 for quantification. If a wireless approach is used, flex sensor 108D might transmit its readings via a low-power radio protocol, thus eliminating the need for a dedicated sensor wire 110. Still, if a wired design is selected, sensor wire 110 may be routed along the glove's underside or along the user's arm to minimize tangling.

Flex sensor 108E refers to the flex sensor positioned on the pinky finger. Although the pinky may appear to contribute less to overall grip force, it is important for balancing and stabilizing the hand during tasks such as holding cylindrical objects, typing on keyboards, or playing musical instruments. By deploying flex sensor 108E along the pinky, the system can register subtle flexion angles that might signal the onset of fatigue or underscore improvements in hand dexterity post-injury.

Like the other flex sensors, flex sensor 108E can be manufactured as a flat, flexible substrate embedded with conductive or resistive elements that vary their electrical properties based on bending angles. The sensor communication board 106 can interpret these electrical changes, optionally performing preliminary analytics. In some aspects, flex sensor 108E′s data may be used to diagnose conditions, where patterns of limited flexion or abnormal extension can be identified over time and enable medical professionals to tailor therapeutic regimes.

As previously described, the sensor communication board 106 may consolidate the electrical signals from each flex sensor (e.g., 108A-108E), condition them (e.g., through signal amplification or filtering), and then transmit the processed data to a higher-level computing device. Depending on the configuration, the sensor communication board 106 can feature dedicated analog-to-digital converter (ADC) channels for each sensor or rely on a multiplexing scheme. I some aspects, the sensor communication board 106 can also coordinate wireless connectivity for the sensors, offering a seamless user experience with minimal wiring.

As previously described, although FIG. 2B highlights sensor wire 110 near the thumb flex sensor 108A, it should be understood that each of the remaining flex sensors may also utilize its own dedicated wiring or share a multi-conductor cable. In one aspect, sensor wire 110 can be a flexible, lightweight ribbon cable that terminates in a connector seated on the sensor communication board 106. In some aspects, each flex sensor 108A-108E may include a small Bluetooth® or similar radio module, circumventing the need for a physical sensor wire 110.

In some aspects, a flex sensor portion 202A-202N may be one or more discrete segments or sensing regions on each finger-specific sensor 108A-108E. These segments can be strategically placed to measure bending at different joints (e.g., the distal interphalangeal joint, proximal interphalangeal joint, and metacarpophalangeal joint), capturing a fuller picture of finger motion than a single sensing element could provide. By parsing data from various sensor portions 202A-202N along a single finger, bending near the fingertip can be distinguished from bending closer to the palm.

In some configurations, sensor portions 202A-202N are realized by printing or depositing multiple resistive tracks in parallel, each track dedicated to a distinct zone of the finger. The sensor communication board 106 can read each zone's electrical signals independently, thus enabling joint-by-joint angle measurement. Where a single continuous sensor strip is used, partial segmentation might be achieved by analyzing the sensor's resistance profile along its length. Such multi-point detection may be beneficial in clinical diagnostics, for instance, revealing whether a certain joint is experiencing restricted movement due to injury or neurological deficits.

In some aspects, flex sensors, at their core, are devices that alter their electrical properties (e.g., commonly resistance) in proportion to how much they are bent or deformed. In some aspects, flex sensors feature a substrate made of a flexible polyester or polyimide film onto which a thin resistive material is deposited. When the sensor is flat, it exhibits a baseline resistance. As the sensor is bent, microfractures or deformations within the resistive layer cause a change in the conductive pathway, typically resulting in an increased resistance. By measuring this change, one can infer the curvature or angle of the bend. Some flex sensors rely on a single resistive element spanning the entire length of the device, while others are segmented into multiple sensing regions (like 202 in FIG. 2B) for multi-joint analysis. Integration with the rest of the system usually involves pairing each sensor with a voltage divider or specialized circuit that tracks the sensor's resistance in real-time. The resulting signal can be digitized, often by an ADC on the sensor communication board 106, enabling software to interpret the bend angle numerically. Calibration procedures can match specific resistance values to known angular displacements, allowing the system to produce consistent readings across different usage scenarios or sensor replacements.

FIGS. 2C and 2D illustrate exemplary implementations of a glove-based sensor assembly 204 configured to capture various biomechanical parameters from a user's hand. In certain aspects, the glove-based sensor assembly 204 is configured to fit over the user's fingers and palm, encapsulating one or more sensor elements such as the FSR sensors 112A-112E or flex sensors 108A-108E. By integrating these sensors into or onto a flexible fabric or polymer material, the glove-based sensor assembly 204 can maintain consistent contact with each finger and fingertip, improving the fidelity of the acquired signals. Referring first to FIG. 2C, a set of FSR sensors 112A-112E are positioned around the distal regions of the thumb, index, middle, ring, and pinky digits. For instance, FSR sensor 112A may capture applied force at the thumb tip, while FSR sensors 112B-112E capture similar force measurements at the respective fingertips of the other digits. In some embodiments, the glove-based sensor assembly 204 can incorporate additional force sensors along intermediate phalanges or the palm, allowing broader coverage of grip pressure distribution. In some aspects, FSRs 112A-112E can be implemented within the glove-based sensor assembly 204 or printed directly onto a flexible substrate within the glove structure. In some aspects, one or more of the FSRs 112A-112E may be arranged between the exterior surface of the glove-based sensor assembly 204 and the skin of a corresponding finger.

In FIG. 2D flex sensors 108A-108E are arranged along the dorsal or lateral surfaces of each finger. Each flex sensor 108A-108E can measure bending angle or changes in curvature, translating mechanical deformation into an electrical signal. For instance, 108A extends across the user's thumb, while 108B-108E tracks flexion in the index, middle, ring, and pinky fingers. Because glove-based sensor assembly 204 ensures that each sensor remains securely oriented against the finger's extensor surface, the system can reliably detect incremental changes in joint angle as the user moves or grips an object. By aggregating these force and flex readings, the glove-based sensor assembly 204 can generate a data profile reflecting the user's overall hand function-useful for tasks ranging from rehabilitation to user interface control. In some aspects, one or more of the flex sensors 108A-108E may be arranged between the exterior surface of the glove-based sensor assembly 204 and the skin of a corresponding finger.

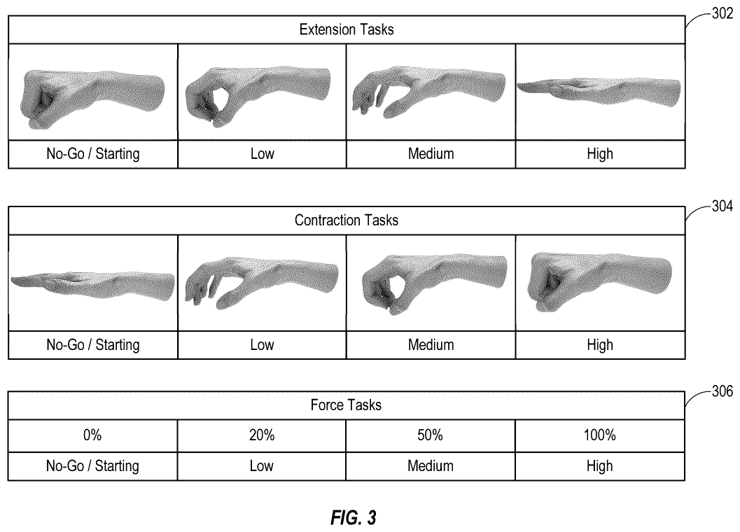

Referring now to FIG. 3, an exemplary illustration of three different categories of motion or force tasks are depicted. In some aspects, these tasks may include extension tasks 302, contraction tasks 304, and force tasks 306. Collectively, these tasks demonstrate how a user can transition from a minimal state (sometimes referred to as “no-go” or baseline) toward higher levels of movement or applied pressure—also described herein as “graded motion.” Graded motion, in this context, involves systematically progressing from a relatively low or neutral starting position (e.g., a fist or a flat hand) to increasingly more challenging positions or force levels. This approach enables sensors (such as flex sensors or FSRs, described in previous figures) to capture data points that reflect variations in the user's hand function. By analyzing these variations, a system can distinguish between different levels of hand extension, contraction, or force application, thereby delivering accurate assessments of user performance or rehabilitation progress.

The extension tasks 302 are depicted as progressing from a no-go or starting position, often a fist or partially clenched hand, to a fully extended or “flat” hand posture. In one illustrative approach, a user begins with fingers curled into a loose fist (the no-go state), remains in that position briefly, and then gradually unfolds the fingers until they reach a target extension angle. In some aspects, a “low” extension might be only a slight opening of the fist, while a “high” extension would approximate a fully open palm with the fingers splayed or nearly so. By sequentially moving through these extension checkpoints, a user performs graded motion, allowing one or more attached (or optical) sensors to capture incremental changes in joint angles.

From a validation perspective, extension tasks 302 can provide structured movements to evaluate sensor measurements against reference standards like those obtained from the optical device 120 (FIG. 1). By quantifying extension at one or more defined steps, an accuracy and reliability can be verified across different movement ranges. For instances, measuring progression from a fist to a partially open hand allows comparison between sensor readings and “ground truth” measurements obtained from the optical device 120 for example. By pairing extension tasks 302 with a calibration process (described further below), each user's unique baseline and maximum extension points can be logged. Such individualized calibration helps to ensure that the sensors can accurately detect and measure changes in extension across the full range of motion.

In some implementations, extension tasks 302 may be varied by altering the wrist or arm position, thereby challenging users to maintain or increase finger extension under different angles or gravity loads. Each variant generates a new dataset that may complement the primary assessment, creating a more comprehensive profile of the user's extension capabilities and uncovering nuances in their hand mechanics.

Contraction tasks 304 may provide the opposite function of extension tasks 302. Rather than opening the hand from a fist to a flat posture, contraction tasks 304 may move from a flat or open palm (the no-go or starting position) toward a high degree of contraction, such as forming a tight fist. In this graded motion, users might progress through intermediate phases, for example, lightly curling the fingers, then forming a half-fist, and ultimately closing the hand as tightly as possible without pain or strain.

In some aspects, these contraction tasks 304 can be used to understand the user's ability to grip or hold objects. By measuring incremental changes in flexion angles at each stage, the system can detect subtle deficits that might indicate problems with muscle strength, motor coordination, or joint flexibility. In therapeutic contexts, contraction tasks can be repeated at varying speeds or under different conditions (e.g., holding a light object vs. performing a purely volitional contraction) to reveal how environmental factors influence the user's movement. The data captured during contraction tasks can be combined with the signals from other figures' sensors, such as force-sensitive resistors or EMG electrodes, to identify whether a user's grip strength aligns with their expected range of motion.

In some aspects, a calibration process also plays a role in contraction tasks 304. For each user, a baseline “flat-hand” measurement may be obtained, and a maximum “full fist” measurement can be established. Intermediate points, e.g., low or medium contraction, can then be defined and stored in the system's memory or software. By mapping real-time sensor outputs to these calibration points, the system can provide immediate feedback on how closely the user's current contraction level matches a target. This feedback loop can support goal-oriented exercises: for example, the user might see a visual indicator (like a bar or color scale) rise toward the “medium” target and can adjust their effort accordingly. As progress is made, the calibration might be updated to reflect new maximums, ensuring that the difficulty level remains aligned with the user's current abilities.

Force tasks 306 may refer to tasks in which the user transitions from applying a low level of force to a comparatively high level of force—often measured through force-sensitive resistors or load cells incorporated in the system. Unlike extension or contraction tasks, which focus primarily on a joint angle or hand posture, force tasks emphasize the pressure exerted by the fingers. In a typical sequence, the user is instructed to press to a specific amount of force against a sensor or object (e.g., a load cell) to establish a maximum force reading (high), then 50 and 20 percent of the recorded sensor measurement is calculated for medium and low. The No-Go or starting force is when no force is being applied to the sensor or by the fingertips. This form of graded motion allows the system to discriminate among different levels of applied pressure and to calibrate those readings against individualized baselines.

Such force tasks 306 may be beneficial in clinical scenarios where strength and endurance are evaluated. By mapping a user's low, medium, and high force outputs onto numerical scales, clinicians can quantify improvements in grip strength over time or detect asymmetries between different hands or fingers. Moreover, force tasks can be integrated with extension and contraction tasks to provide a multi-dimensional profile of hand function. For instance, some protocols might require the user to maintain a specific hand posture (e.g., partial fist) while applying gradual increments of force, revealing whether the user can sustain an isometric contraction without fatigue or compensatory postures.

During a calibration phase for force tasks, each user may undergo a short set of reference presses, thereby defining a no-go (or negligible force) state and a maximum force state they can comfortably achieve. Intermediate target forces—e.g., 25%, 50%, or 75% of the maximum—are recorded. The sensor's output at each level can then be stored to form a calibration curve. Consequently, when the user performs force task 306 in real-time, the system can interpret the raw sensor data in relation to this individualized curve, providing immediate biofeedback on the magnitude of force being generated. This approach may be valuable in rehabilitation contexts, where safe and consistent progression may be key to preventing overexertion or reinjury.

Across extension tasks 302, contraction tasks 304, and force tasks 306, calibration ensures that the system's sensors accurately reflect each user's unique capabilities and biomechanical ranges. In one exemplary procedure, the user first performs no-go or baseline positions—such as a relaxed fist for extension tasks or a flat hand for contraction tasks—while the sensors record baseline readings. Next, the user is asked to reach a comfortable maximum (e.g., fully extended hand or fully clenched fist), and these readings are similarly captured. For force tasks, low-pressure (negligible force) and high-pressure (user's maximum safe force) references are taken. The software or firmware running on the system then uses these values to define a mapping between raw sensor outputs (like voltage or resistance) and meaningful human-readable levels (e.g., “Low Extension,” “Medium Contraction,” “High Force”).

Once calibration is complete, subsequent movements or force applications can be interpreted in real-time against this personalized scale. The system can thus provide more finely tuned assessments or feedback, recognizing, for instance, that a certain user's “medium extension” is numerically different from another user's due to differences in anatomy, strength, or flexibility. If the user's range of motion or maximum force changes over the course of therapy or training, the calibration process can be repeated to ensure that all reported measurements remain accurate and clinically useful.

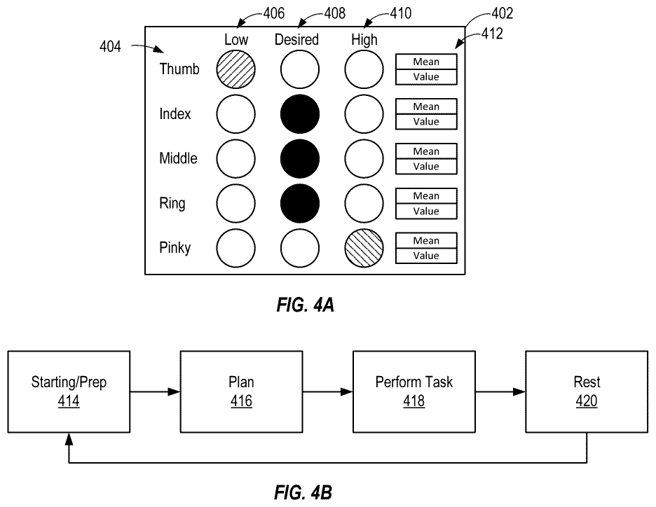

Referring now to FIGS. 4A and 4B, an exemplary user interface for guiding individuals through various hand-related tasks or assessments, as well as a general workflow depicting how such an interface and tasks may be carried out, are depicted in accordance with aspects of the present disclosure. In FIG. 4A, GUI 402 presents distinct indicators corresponding to finger identification labels 404 (e.g., “Thumb,” “Index,” etc.) and provides real-time, or near real-time, visual feedback of sensor measurements to users during task performance. Each finger can be associated with target categories—“Low” 406, “Desired” 408, and “High” 410—enabling a user or clinician to set specific performance thresholds. Additionally, GUI 402 provides mean or actual values of 412 to facilitate real-time or post-hoc analysis of results. In FIG. 4B, a flow diagram illustrates phases (414, 416, 418, 420) of a typical session, from preparation and planning through task execution and rest.

In some aspects, the GUI 402 may be rendered on a screen, tablet, or other display medium. A GUI 402 may be programmed to show real-time feedback derived from sensor measurements (e.g., flex sensors, force sensors, or other biomechanical sensors) so that participants or clinicians can gauge performance at a glance. The GUI 402 may include graphical elements such as circles or bars representing each finger's status relative to preset thresholds (for instance, the “Low,” “Desired,” and “High” labels described below). In some aspects, GUI 402 dynamically updates in real-time, visibly shifting color or size to highlight whether a user's current hand movement or force output meets, exceeds, or falls short of the defined targets. During calibration procedures, the GUI 402 might prompt the user to assume a specific hand posture or apply a specific amount of force, then log the sensor readings for future comparisons. In a rehabilitative context, GUI 402 can be used both by the individual performing the tasks and by medical personnel overseeing therapy sessions—each needing quick, intelligible feedback on the user's progress. Different interface modes could be implemented, one for novice users that only shows simplified indicators (e.g., “OK” vs. “Needs Improvement”) and another for advanced users or clinicians offering more granular metrics. By consolidating all relevant data—finger labels, threshold categories, and mean/actual values—GUI 402 becomes a central hub of engagement throughout a training or assessment session.

Finger identification labels 404 may be visual or textual markers denoting which finger is being measured or displayed in GUI 402. Common labels include “Thumb,” “Index,” “Middle,” “Ring,” and “Pinky,” though the system could accommodate additional references for specialized or customized usage. These labels help the user pinpoint exactly which digit's performance is being tracked, facilitating more targeted feedback and training. Each label 404 may be placed adjacent to or within a corresponding row or column on the interface, ensuring clarity about which sensor data belongs to which finger. For instance, in a scenario where a person is trying to achieve a “Desired” force level with the index finger, GUI 402 may highlight the row labeled “Index” while displaying the relevant threshold circles or bars. In some systems, labels 404 can also be color-coded or dynamically lit when a particular finger is active, such as blinking or changing color if the system detects motion or force in that digit. By making finger identification explicit, the system reduces confusion, especially if multiple digits are being used simultaneously or if the user has partial impairments that make certain fingers harder to control. Such labeling further enables clinicians or observers to annotate records with finger-specific notes (e.g., “Index finger shows improvement” or “Ring finger needs extra exercises”) for subsequent review.

In some aspects, the low label 406 may be used to gauge whether a user's contraction or extension remains below a designated minimal level. Within a single exercise, for instance, a graded contraction task, the system can instruct the user to contract their hand to a low contraction task (e.g., aiming for 20%). The graphical GUI 402 displays whether the actual measured value (e.g., flex angle or force reading) for each finger meets the desired measurement, falls short (e.g., low), or surpasses (e.g., too high). Thus, the low label 406 represents a category or band of exertion (or movement) that the user should strive to be above for the designated portion of the exercise.

The desired label 408 indicates a more ideal threshold, often being the primary target for a particular graded task. Continuing the graded contraction example, 408 may represent an angle that is considered to be the most desired angle for the specific graded task. The GUI 402 can visually flag when each finger's measured values align with this desired band, indicating the user is applying just the right amount of effort and, more so, the finger moves a desired amount. By monitoring how long the user stays within, clinicians gain insight into muscle endurance and coordination. If the user overshoots or undershoots (ending up in the low or high zones), the graphical GUI 402 promptly signals that adjustment is needed.

The “mean value” or the ongoing “actual value” may be displayed alongside the threshold indicators in GUI 402. This numeric or textual data typically shows the average and/or the current sensor reading for a particular finger or performance parameter. For instance, if the user is applying variable force with the thumb, the interface might display the real-time force reading in Newtons or a relative scale while also computing an average (mean) value over a set time window. The mean value can help smooth out transient spikes or dips, offering a clearer picture of the user's sustained performance.

Meanwhile, the actual value may be useful for immediate feedback, showing the user whether they are moving closer to or further from the “Low,” “Desired,” or “High” thresholds. This real-time data can be updated multiple times per second, depending on the application's needs for responsiveness. In a calibration phase, these numerical displays confirm that the system has properly recorded baseline and maximum sensor readings. During active assessments, the user can glance at the mean or actual value to gauge consistency—for example, determining if they are holding a steady contraction or if their force is fluctuating. Over multiple sessions, 412's data can be logged, enabling therapists or users to identify trends in improvement, plateaus, or regressions. By coupling numeric values with the threshold indicators, GUI 402 provides both qualitative (visual target zones) and quantitative (mean/current readings) insight into performance. Of course, one or more of the items depicted in the graphical GUI 402 may be present or not present.

When performing the graded extension task depicted in FIG. 3, participants might see the GUI 402 instruct them to hold a “Low” angle for a period of time (e.g., 5-8 seconds), then return to No-Go (rest). Conversely, in graded contraction, the interface may shuffle (at random) through “Low,” “Medium,” or “High” angles for each task and/or each finger for a period of time. For force tasks, the system similarly designates Low, Medium, and High target forces for the thumb, index, or ring finger, with repeated trials to assess consistency. Because these tasks are repeated many times within an action set, and each action set is performed a certain number of times, the interface helps participants systematically cycle through instructions—e.g., “Contract Index Finger at Medium,” or “Perform the Depicted Task,” or “Press Load Cell at High Force”—while logging how accurately they match the assigned threshold. In so doing, the GUI 402 helps to standardize data collection and validation.

In some aspects, FIG. 4B depicts a process for performing a graded motion task in accordance with aspects of the present disclosure. In some aspects, a user may start at a starting or preparation phase 414, where users ready themselves for the upcoming task or assessment. During starting or preparation phase 414, the system may display instructions on GUI 402, prompting the user to take a neutral hand posture (e.g., a relaxed fist or open palm) or to ensure the sensors are correctly positioned. If a force sensor is involved, the user might be asked to apply zero or minimal force so that the system can establish a baseline reading.

Clinically, starting or preparation phase 414 serves to focus the participant's attention, typically using a fixation cross displayed for one to two seconds before the task begins. The interface may include a progress bar that shows participants how many prompts and targets remain in their current run. Before initiating the GUI 414, the operator can verify proper sensor function and system setup.

In some aspects, the plan phase 416 in FIG. 4B may commence once the user is prepared; accordingly, the system or clinician formulates a plan for the specific task or sequence of movements to be performed. For instance, if the session involves graded extension exercises, plan phase 416 might define the exact sequence (e.g., from “Low” extension to “Medium” extension, repeated three times). Alternatively, if the task focuses on force, plan phase 416 would specify the target thresholds—say, low force, then medium force, then a high force hold.

During this planning phase, GUI 402 may present an outline of the upcoming tasks. The user can review or confirm the steps, ensuring clarity before moving on to the “perform task” phase. In scenarios that involve multiple fingers, plan phase 416 might break down the sequence finger by finger or present an interleaved approach (e.g., alternating index and middle finger tasks). If the user is in a clinical environment, the therapist might modify the plan in real-time based on the user's baseline performance or overall condition. For example, if the user appears fatigued, the plan might reduce the number of high-force trials. Conversely, if the user performs well during calibration, the plan might be made more challenging. Plan phase 416 thus ensures that every session is systematic, goal-oriented, and adjustable, laying out a clear roadmap for both the user and the supporting medical or training personnel.

Following the plan phase 416, the user carries out the designated motions or applies the instructed force levels. During this phase, GUI 402 delivers real-time feedback, for instance, by lighting up the relevant “Low,” “Desired,” or “High” indicators or displaying numeric mean/actual values 412. If multiple fingers are involved, the interface may highlight each finger label 404 in turn, guiding the user step by step.

In some aspects, the performance task phase 418 phase is the core of the assessment or exercise session, capturing data about the user's ability to meet and maintain specified thresholds. In some embodiments, the user might be directed to hold a “Desired” force level for a set duration, while in others, the system might instruct them to cycle between different graded tasks (e.g., low, medium, high). The sensors (e.g., 108 and 112 of FIG. 1) may continuously gather raw signals, which can then be interpreted by the software to update the GUI 402. This dynamic loop helps the user self-correct at the moment—for example, noticing if they are drifting from the “Desired” zone into “Low” or “High.” The data recorded here can be stored locally or transmitted to a remote server, enabling subsequent analysis. Overall, the perform task phase 418 transforms the planned protocol into actionable movements, each of which is closely tracked and evaluated to measure progress, identify weaknesses, and inform future calibrations.

Once the user completes a round of tasks, they transition into a rest period for recovery and data reflection. During rest phase 420, GUI 402 may display summary statistics (e.g., the average force or angle, how often the user stayed in the “Desired” zone, etc.), allowing both the user and clinician to review immediate outcomes. Additionally, if the system is configured for multiple repetitions or sets, rest phase 420 offers an interval to reset physically and mentally before returning to preparation phase 414.

The duration of this rest can be adjustable, depending on the user's condition or the complexity of the tasks. For instance, a therapist or clinician might recommend longer breaks for patients with significant fatigue issues, ensuring they do not risk injury or discouragement. Alternatively, shorter rests may be appropriate for higher-intensity training scenarios. During rest phase 420, the interface might also suggest mild stretching or relaxation techniques to prevent stiffness in the hand. If any performance anomalies were noted in the preceding “perform task” phase, rest 420 is an opportunity to address them—perhaps adjusting sensor placement or fine-tuning the next iteration's thresholds. Once the rest period concludes, the user can circle back to preparation phase 414 for another round, continuing through the sequence outlined in FIG. 4B. The rest duration can be adjusted based on task complexity and assessment needs.

GUI 402 is employed both during calibration and throughout actual assessment exams to guide and inform the user. During calibration, the interface typically presents a series of prompts or targets—such as “Low,” “Desired,” and “High”—for each labeled finger. The user follows on-screen instructions, for instance, by making a relaxed fist or applying minimal force to establish the baseline. The interface records sensor readings at these defined positions or force levels, enabling a personalized mapping between raw sensor data and meaningful performance metrics. This calibration step ensures the system can recognize subtle variations in each finger's movement or force output, accounting for individual differences in hand size, strength, and flexibility.

During an assessment exam, the GUI 402 can display real-time feedback. For each finger, the system highlights whether the user is currently in the “Low,” “Desired,” or “High” zone. Visual cues—such as color changes or icons—help the user instantly see if they are meeting the specified criteria for extension, contraction, or force. Concurrently, mean or actual values (412) appear alongside the thresholds, offering numeric confirmation of the user's performance. These readings update continuously, enabling the user to adjust their posture or force as needed.

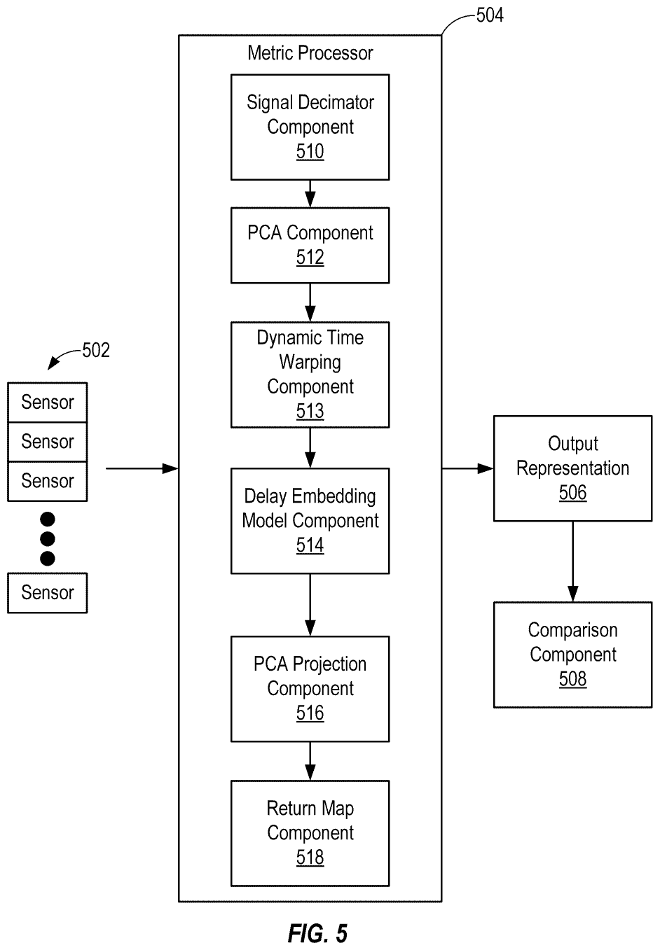

FIG. 5 depicts an exemplary signal analysis and processing flow for evaluating user hand-movement data obtained from one or more sensors 502 and deriving clinically relevant metrics therefrom. In some aspects, a metric processor 504 orchestrates several modular components, including but not limited to a signal decimator component 510, principal component analysis (PCA) component 512, dynamic time warping component DTW component 513, delay embedding model component 514, PCA projection component 516, and return map component 518—to generate an output representation 506 and enable comparisons against normative data (via comparison component 508). While depicted in a particular sequence, the processing blocks of the metrics processor 504 may be performed in different orders, and some blocks may be optional depending on requirements of one or more analyses and sensor data characteristics. By using dimensionality reduction, time-alignment, and state-space transformations, the system can detect subtle differences in hand function, compare user performance to unimpaired references, and generate clinically meaningful feedback for rehabilitation or diagnostic purposes.

As previously described, a suite of sensors configured to capture physiological and biomechanical signals from a user's hand or arm may refer to flex sensors 108 and FSR sensors 112. Such sensors may include FSR sensors, flex sensors, and other sensors, such as but not limited to, inertial measurement units, electromyography (EMG) electrodes, or optical trackers. Collected signals can reflect finger joint angles, applied grip force, muscle activation patterns, or overall arm posture. Sensor data may be sampled at high frequencies to capture rapid transitions—such as moving from a relaxed hand to a fully clenched fist—but the specific sensor count and placement may vary by clinical objective.

In practice, raw signals from sensor 502 may be digitized in real-time or recorded for offline analysis. For example, a stroke-rehabilitation setup could rely on multiple force and flex sensors across the fingers to map individual digits' movements and forces during grasp-and-release tasks. Sensor data from sensors 502 serves as the foundational data, feeding measurements into the metric processor (504) for subsequent interpretation and feedback generation.

In some aspects, the metric processor 504 transforms incoming sensor data (e.g., sensor data from sensor 502) into clinically actionable metrics. The metric processor 504 may include various components, each performing a distinct function in the analysis pipeline. In a rehabilitation context, a metrics processor 504 could run continuously, providing near real-time feedback to a patient or clinician; alternatively, it can operate in batch mode for offline studies.

The signal decimator component 510 may resample, filter, or otherwise reduce the volume of incoming data from sensors 502. In high-sampling-rate scenarios, raw signals may be unnecessarily dense, complicating real-time analysis and inflating computational costs. Accordingly, signal decimator component 510 can apply smoothing filters or down sampling rules that preserve essential motion features while discarding extraneous high-frequency content.

The PCA component 512 may identify the dominant variance directions (principal components) across multiple sensor channels. In some aspects, the PCA component 512 may distill the raw signals into a handful of orthogonal components that succinctly characterize the user's hand function. For instance, one principal component might capture overall finger-curling movement, while another focuses on thumb rotation or force application.

In some aspects, a PCA can expose patterns that discriminate between unimpaired and impaired motor function. By working in a reduced component space, the system also mitigates noise and redundancy in large sensor arrays. These PCA outputs feed-forward to DTW component 513 to facilitate advanced time-series alignment or state-space transformations.

While PCA reduces dimensionality, real-world motion signals can still be out of phase or vary in duration across different trials or different users. DTW component 513 addresses that mismatch by time-warping (or otherwise aligning) the PCA-reduced signals so that similar motion phases coincide more closely. For example, if Participant A flexes and extends faster than Participant B, their raw signals might look offset or compressed in time. DTW component 513 can automatically stretch or compress the sequences so each key event (e.g., peak contraction) lines up in both signals. The DTW component 513 may provide a uniform time basis, ensuring that subsequent analyses, like the delay embedding model component 514, are not confounded by arbitrary pacing differences. In some aspects, the delay embedding model component 514 constructs a phase space from the aligned low-dimensional signals output by the DTW component 513. In some aspects, repeated or cyclic tasks—such as repeatedly opening and closing the hand—produce loop-like trajectories that reveal consistency, stability, or deficits in motor control. For clinicians and researchers, these delay-based phase spaces help detect subtle changes in how a user executes each movement cycle. A stroke patient's inconsistent or incomplete motions might manifest as irregular loops or chaotic trajectories, whereas a non-impeded subject performing the same task could produce clean, stable loops. By applying the delay embedding model component 514 after time alignment in 513, the resulting trajectory more accurately reflects each user's intrinsic motor pattern, unskewed by differences in speed or timing.

In some aspects, after the signals have passed through delay embedding model component 514, PCA projection component 516 can further refine the representation for visualization or machine-learning steps. During PCA projection, the PCA projection component 516 may update principal component mappings to the newly formed, particularly if the phase space itself is still somewhat high-dimensional.

Within a rehab context, if repeated tasks generate repeated loops in an embedded space, the PCA projection component 516 clarifies whether those loops differ primarily in amplitude, shape, or consistency. In some aspects, the PCA projection component may provide an indication as to which finger is dominant during which task.

In some aspects, a return map component 518 may generate a return map revealing cyclical or quasi-periodic structures. For tasks involving repeated grasp-release cycles, a non-impeded subject might create compact, well-defined loops, whereas an impaired individual's return map could exhibit scattered, overlapping loops, signaling difficulties with consistent movement execution.

By comparing these return maps over time or across subjects, clinicians can identify whether a patient is improving or if further intervention is needed. The return map may be especially good at highlighting subtle disruptions in rhythmic tasks (e.g., tremor, spasticity), which may not be obvious from raw signals alone. Because the return map component 518 can be integrated with the comparison component 508, a system can quantify how close a user's return-map loops are to a normative pattern or to the user's own baseline from a previous session.

The output representation 506 may transform the processed signals-whether in return-map form, time-aligned waveforms, or principal-component trajectories, into human-readable graphs, dashboards, or numeric scores. Clinicians and users alike can then visualize the user's performance and progression. The comparison component 508 may leverage these outputs to assess how a user's data compares to normative references or personal baselines. If the comparison reveals subpar performance on pinch strength yet near-normal finger extension loops, the system or a clinician might recommend targeted therapy focusing on pinch exercises.

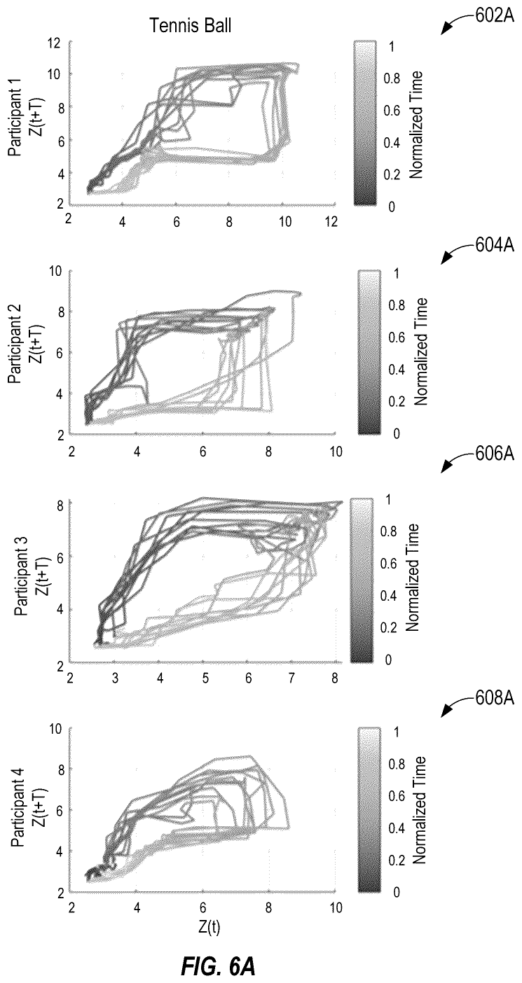

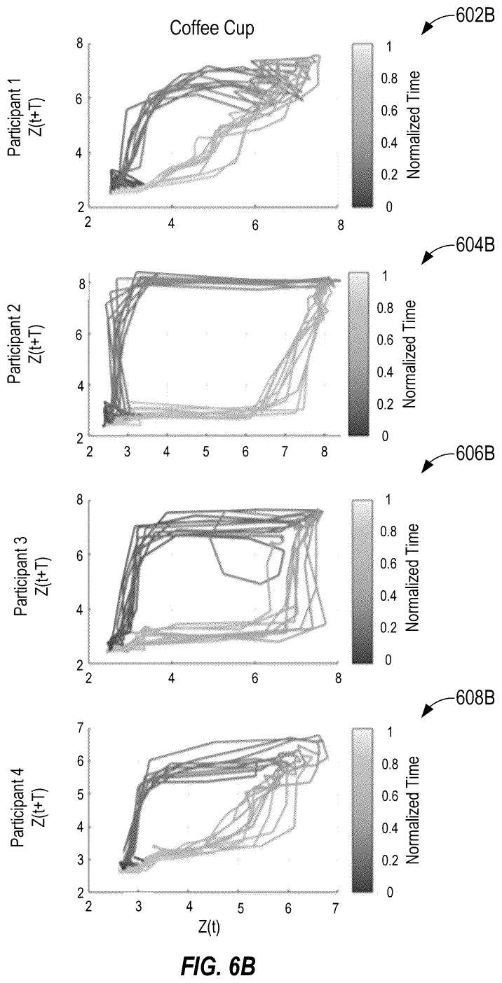

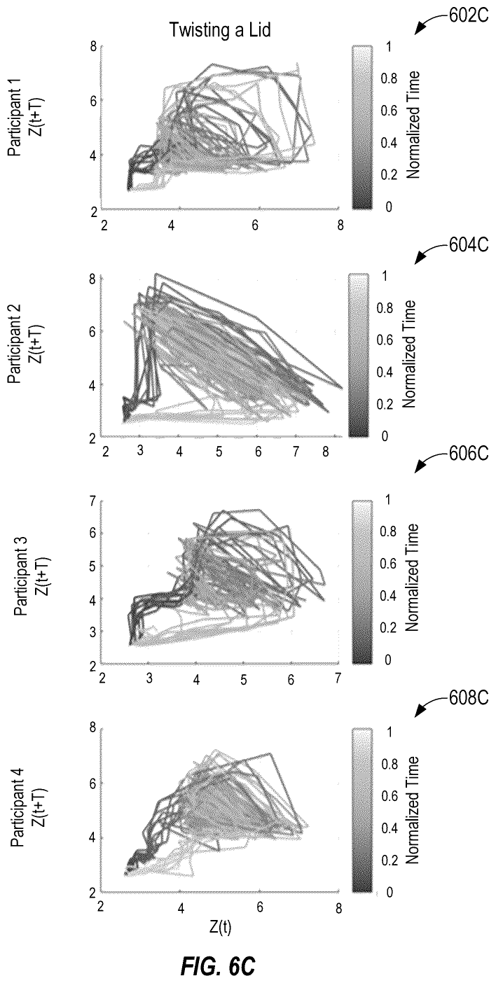

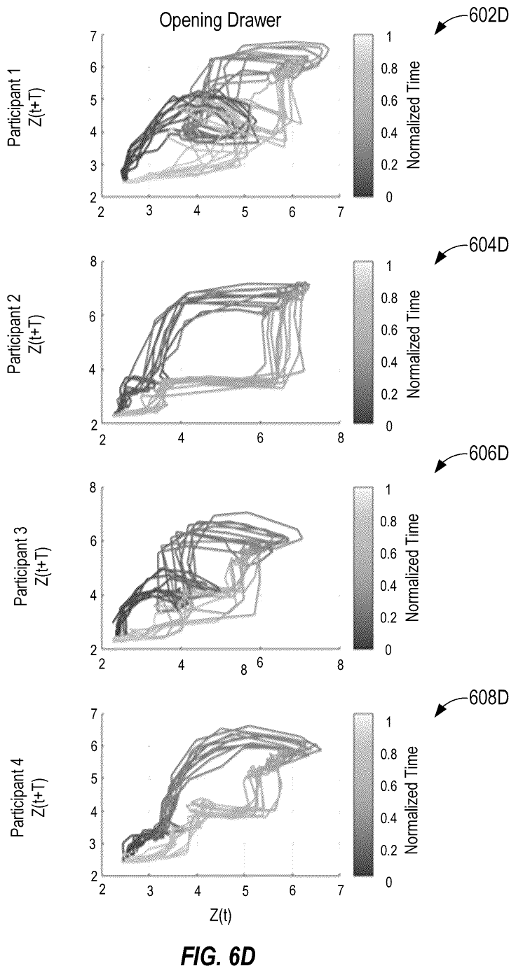

FIGS. 6A-6D depict exemplary return maps for multiple participants performing four distinct hand-related tasks. Each sub-figure presents a series of trajectories, where the horizontal axis (Z(t)) and the vertical axis (Z(t+τ)) represent time-lagged components of a reduced-dimensional signal (e.g., from principal component analysis) associated with finger or hand movements. The grayscale bar adjacent to each plot indicates “normalized time,” enabling viewers to trace the participant's progress from the start of the task (darker shades) to its conclusion (lighter shades). The tasks illustrated, tennis ball grasping, coffee cup manipulation, twisting a lid, and opening a drawer, are representative of functional hand activities. By comparing differences in loop shapes, overlaps, or the spacing of trajectories within each return map, clinicians and researchers can identify how consistently or effectively each participant performs the relevant motion. In various implementations, these return maps may be derived using PCA and delay embedding to visualize the motion patterns. Descriptions of each reference character follows.

Reference character 602A represents a return map associated with a first participant's performance of the “tennis ball task.” In certain implementations, the tennis ball task involves instructing the user to pick up a tennis ball from a flat surface, hold it in a stable grip, possibly rotate it or move it, and then release it back onto the surface. This exercise is often chosen for rehabilitation or diagnostic assessments because it requires coordinated finger extension and flexion, combined with moderate grip force to prevent the ball from slipping. In the return map of 602A, each loop corresponds to a single “grasp-and-release” cycle, with lighter gray shades indicating the start of a cycle and darker gray shades near its end. A smoothly formed loop may signify steady or consistent movement, whereas irregular shapes or overlapping lines can highlight tremors, inexact grip control, or inconsistency between repetitions. Clinicians can compare the geometry of the loops in 602A to normative loops for non-impeded individuals, thereby pinpointing any functional deficits or improvements over time. Further, the presence of multiple loops can indicate repeated attempts by the participant, allowing clinicians or therapists to observe if performance stabilizes across successive trials. By examining the transitions in normalized time along the trajectory, it becomes feasible to understand precisely when the participant applies force, adjusts grip, or initiates the release phase.

Reference character 604A pertains to a second participant's return map of the tennis ball task, capturing how a different user's movements unfold in the same exercise. Although the fundamental instructions-pick up the tennis ball, manipulate it, and place it back-may be identical, each participant exhibits unique motor patterns and timing. The return map in 604A thus allows direct visual comparison to 602A: for instance, one might notice that the loops in 604A are more compressed or more spread out, indicating differences in the range of motion or the time spent in certain grip postures. If the participant represented by 604A has difficulty maintaining a stable grasp, the loops could show irregular or jagged edges, suggesting frequent micro-adjustments or muscle fatigue. In a clinical setting, tracking how the shape of these loops evolves over multiple sessions can reveal whether the individual is developing more efficient motion control-for example, loops might become more uniform or shift toward an ideal region of the map. Furthermore, the normalized time gradient provides additional context: if the participant hesitates mid-task, there might be a denser cluster of lines in that region, visually highlighting where the user slows or pauses. Such information can guide therapists to recommend targeted interventions, such as finger-strengthening drills or improved hand placement strategies.

Reference character 606A corresponds to yet another (third) participant's tennis ball return map, offering a third perspective on the same functional activity. Distinguishing aspects within 606A might include fewer loops (if the individual performed fewer trials), a narrower range in Z(t) or Z(t+τ) coordinates (indicating more limited flexion-extension amplitude), or longer transitions in normalized time that point to slower overall execution. By examining whether the participant's loops overlap consistently or spread out haphazardly, clinicians can assess the degree of motor planning and control each trial exhibits. If the participant demonstrates partial completion of the exercise-such as picking up the ball but struggling with the release loop could terminate prematurely or deviate drastically from the typical cyclical pattern. Moreover, researchers can compute metrics such as loop area, loop eccentricity, or average trajectory length from 606A to quantify improvement over repeated sessions. In some embodiments, specialized software may color-code the loop segments based on sub-phases (e.g., contact initiation, peak grip force, ball rotation), refining the clinical analysis. By comparing 606A to data from unimpaired volunteers, this system can also gauge the extent to which the participant's motion deviates from normative patterns and whether additional interventions are warranted.

Reference character 608A showcases a fourth participant's tennis ball return map, completing the set of four tennis ball examples in FIG. 6A. Even though the same basic instructions apply, the participant shown in 608A might exhibit entirely different speed profiles, force application timing, or joint coordination strategies, resulting in distinct loop shapes. For instance, if the participant is learning the task for the first time or lacks hand strength, the loops could be less smooth, with abrupt transitions in the normalized-time shading. Alternatively, a participant with extensive therapy behind them could produce loops resembling those of a healthy control subject, indicating near-normal hand function. The utility of 608A thus lies not only in diagnosing current deficits but also in tracking progress: repeated measurements can confirm whether the user's loops become more stable or expand into a healthier range, signifying improvements in grip or finger movement synergy. Combined with an analysis of standard deviation in loop coordinates, 608A can highlight whether the participant is consistent across trials or still variable from repetition to repetition. Hence, this return map is a visualization for clinicians aiming to isolate specific motion deficits-such as limited finger extension or inconsistent release patterns-under realistic manipulative tasks involving everyday objects like a tennis ball.

Reference character 602B of FIG. 6B illustrates a first participant's return map derived from performing a “coffee cup task.” In this scenario, the user is instructed to reach for a standard coffee cup (e.g., a bell-shaped ceramic mug), grasp it around its body, lift it, and then return it to its resting place. The required coordination—maintaining a secure grip on the cup's curved surface, adjusting grip force to account for the object's shape, and stabilizing wrist orientation—can test fine motor control in a clinical or research context. The return map of 602B visualizes these cycles, with each loop corresponding to a pick-up and put-down sequence. Variations in loop thickness or the distribution of gray shading along the trajectory can indicate hesitations, re-grips, or instabilities. The trajectory may show repositioning of fingers along the cup's surface as participants adjust their grip. Over repeated trials, analysts can determine whether the participant's technique converges toward a more consistent, streamlined loop, a sign of improving dexterity and grip confidence.

Reference character 604B depicts the coffee cup task performance by a second participant. However, labeled similarly to a reference in FIG. 6A, this instance corresponds to a distinct set of recorded movements for the coffee cup exercise. By comparing the shape of these loops to those from the first participant (e.g., 602B), clinicians can see how the second participant's grip or motion strategy differs. For example, the loops could be taller in the Z(t+τ) axis, implying a higher overall range of hand rotation or bigger amplitude in flexion extension. If the participant occasionally fumbles or sets the cup down uncertainly, the loops might become ragged in those time intervals, with darker clusters of lines indicating repeated corrections. Importantly, the coffee cup task is reminiscent of real-life daily living; the correlation between the smoothness of the loop and the participant's actual day-to-day functional independence can be more direct than in purely synthetic tasks. By monitoring trends in 604B across sessions—whether the loops simplify or the transitions even out—therapists glean objective evidence of the user's progress in a crucial daily skill (handling cups, utensils, or similar objects).

Reference character 606B indicates the third participant's coffee cup return map, further illustrating inter-participant variability in a real-world object manipulation scenario. Each gray loop in 606B corresponds to a single “pick up, simulate sip, put down” cycle. In some embodiments, the amplitude on the horizontal axis might correlate to how far the wrist or arm moves laterally, while the vertical axis could reflect changes in grip or finger flexion angles, all extracted and time-lagged from sensor data. If the loops in 606B appear compressed or barely vary along one axis, it could imply a limited range of motion, possibly due to muscle weakness or post-stroke hemiparesis. Conversely, loops that form large arcs might indicate robust, sweeping movements, though not necessarily stable or smooth. Analysts can also interpret how the participant's motion evolves temporally via the grayscale shading: abrupt color changes might mark transitions from grip-establishment to cup-lift or from tilt-back to cup-lower. Over repeated attempts, stable, closed loops generally reflect improved motor planning and execution, whereas scattered trajectories suggest ongoing motor control challenges.

Reference character 608B completes the set of four coffee cup return maps by depicting a fourth participant's sequence of loops. Despite identical instructions-acquire the cup, mimic a drinking action, and return it-every participant's motion style and control level can differ significantly. The spacing and layering of loops in 608B may show whether the participant varies each attempt or reproduces a nearly identical pattern each time, indicative of consistent motor learning. Large swaths of overlap might suggest the user follows a very similar motion path for each trial while diverging lines could mean that the user experiments with different grips or compensates for fatigue in later attempts. This plot is especially relevant for identifying subtle improvements or regressions: for instance, if the participant's initial loops were shaky but later loops became more compact and cyclical, that pattern might confirm successful motor retraining. By contrast, disorganized or partial loops might reveal that the participant has trouble controlling the mug's angle or handle, necessitating further targeted rehabilitation exercises to build the necessary finger dexterity or wrist stability.

Reference character 602C in FIG. 6C relates to a first participant's effort to “twist a lid.” This task mimics opening or closing a jar or bottle, requiring coordinated finger flexion, thumb opposition, and forearm rotation. In many rehabilitation protocols, twisting a lid is recognized as a key indicator of wrist and finger synergy, as well as pinch strength. The return map thus logs cycles of partial rotation, re-grips, or full rotations. Particularly in scenarios where the participant must repeatedly re-position fingers to maintain torque, loops may exhibit expansions or irregular detours. A neatly enclosed loop in 602C could imply smooth, confident twisting with minimal hesitation, whereas heavily overlaid lines might suggest frequent slippage or incomplete grips. Additionally, the brightness gradient from 0 to 1 in normalized time can clarify whether the participant moves steadily or if certain time periods exhibit extended dwell (e.g., readjusting the lid). Over multiple sessions, quantitative measures extracted from 602C can demonstrate whether a patient recovering from hand surgery is regaining rotational dexterity or if alternative training is needed.

Reference character 604C in FIG. 6C shows how a second participant approaches the same twisting-a-lid challenge. By comparing the shape and location of loops in 604C to those in 602C, clinicians can evaluate whether the second participant is more or less adept at coordinating wrist rotation with finger grip. For instance, if the participant's loops deviate widely in the Z(t+τ) dimension, it might signal forceful but inconsistent attempts, possibly reflecting the difficulty in sustaining the torque. The presence of partial or truncated loops could indicate repeated “stop and start” intervals, such as if the participant must pause or rest the hand mid-twist. Since real lids often require incremental progress (turn, reposition, turn again), the path might reflect multiple mini-cycles within each main attempt. This can be valuable for highlighting whether the participant's technique is efficient or if they rely on compensatory movements that a therapist may want to correct. Over repeated tasks, improvements in the uniformity of these loops often align with better real-world jar-opening abilities.

Reference character 606C in FIG. 6C depicts a third participant's lid-twisting return map. Each “loop” can represent a full or partial rotation, with the normalized time shading indicating how the participant's motion evolves from beginning to end. In certain implementations, the horizontal axis might represent a measure of the participant's grip tightness or radial-ulnar deviation, while the vertical axis captures forearm rotation angles or a time-lagged synergy of finger movement. If the participant strongly rotates the lid at first but tires quickly, the loops could appear large near the start but then shrink or cluster later on, signifying reduced rotation amplitude. Alternatively, consistently sized loops could mean the participant manages uniform torque throughout multiple attempts. This is often a prime indicator of re-learning fine motor control post-injury or post-stroke. By closely examining how loops in 606C differ from the earlier participants, clinicians might pinpoint individualized deficits such as an overreliance on wrist pronation or difficulties stabilizing the jar with the opposite hand. This knowledge directly informs targeted interventions, possibly recommending forearm-strengthening exercises or improved grip postures.

Reference character 608C in FIG. 6C offers the fourth participant's lid-twisting return map. Even with identical instructions, the participant's personal hand strength, finger dexterity, and arm coordination often yield a unique trajectory. If the loops in 608C appear relatively simple, forming one or two distinct cycles, the participant may accomplish the lid twist in fewer, more deliberate motions. However, if numerous overlapping loops appear, that might mean multiple short attempts-like partial twists or re-positioning the lid-are needed before completion. The color scale can highlight extended dwell times at certain phases (e.g., 40-60% normalized time), possibly correlating to the highest torque demand. Clinically, analyzing this map can help determine whether the participant's challenge stems from insufficient grip force or from poor synergy between the wrist and fingers. Over repeated visits, clinicians can track whether the participant's loops converge toward a neater pattern, reflecting progressive mastery of the twisting task. If not, advanced therapies or specialized braces might be recommended to facilitate stronger or more controlled rotational manipulation.

Reference character 602D of FIG. 6D depicts a first participant's return map for an “opening drawer” task, a maneuver commonly used to evaluate functional extension, pulling force, and grip synergy. In many scenarios, the participant is asked to grasp a drawer handle, apply enough outward force to overcome friction or a latch and slide the drawer open by a certain distance before pushing it back. This demands forearm stability, coordinated finger flexion to maintain handle contact, and a smooth extension or retraction of the arm. In the return map of 602D, each loop generally correlates to an “open and close” cycle. Smaller loops could appear if the participant performs partial openings. The shading from light to dark reveals the timing of each sub-phase-grip acquisition, pull, hold, and release. If the participant struggles with consistent force or alignment, the loops may show abrupt shifts or flatten in places, reflecting hesitation or re-positioning. By studying such patterns, clinicians can determine if further training is needed to develop the finger, wrist, and shoulder synergy integral to everyday tasks like opening cabinet drawers or desk drawers.

Reference character 604D shows how a second participant navigates the opening drawer challenge. Differing loop shapes might arise from the user's personal approach—some individuals lean their body, use two or more quick tugs, or gradually shift their grip during the pull. By mapping these variations as loops in the Z(t) vs. Z(t+τ) plane, professionals can detect if the participant is stable (forming uniform cycles) or inconsistent (producing scattered or partial loops). For instance, a cluster of lines near the midpoint of the loop might indicate a zone of repeated micro-adjustments or stalling. The grayscale bar's range reveals if the participant lingers at certain time intervals-perhaps waiting for the force threshold needed to keep the drawer sliding smoothly. Over successive trials, improvements in loop consistency or expansion of the motion range can confirm positive therapy outcomes. Meanwhile, partial loops or loops that never return to an initial baseline might suggest incomplete motions or enduring difficulties with extension and steady pulling force.

Reference character 606D depicts the third participant's cycles of opening and closing a drawer, again visualized via a return map. Each trajectory loop highlights how the participant transitions from no contact to a firm grip, initiates an outward pull, maintains it, and then reverses the action to push the drawer closed. If the participant's loops are elongated along one axis, it could signify a large variation in how far the drawer is pulled out each attempt, perhaps due to uncertain grip force or inconsistent motor control. Conversely, tight clustering might show a repeatable, practiced motion with minimal variability. Observers can glean when the participant exerts maximum force or re-positions the hand by noting abrupt changes in the loop's slope or coloring. As with the prior references, 606D can be compared to normative data to assess how the participant's motion patterns deviate from typical or normal ranges, enabling objective judgments about progress or the necessity for specialized interventions (like wrist supports or adaptive equipment).

Reference character 608D completes FIG. 6D by showing the fourth participant's opening drawer return map. Even if all participants receive the same instructions, their personal motor abilities, hand strength, and comfort with the handle can result in distinctive loop configurations. In some cases, a participant might produce smaller, consolidated loops if they consistently open the drawer only partially. Alternatively, widely spread loops may indicate repeated pushing-pulling cycles as the participant attempts to find the correct angle or force level. The color gradient tracking normalized time can also reveal whether transitions are swift or if the participant dwells between phases, for example, taking a moment to readjust the grip. In ongoing therapy, repeated recordings might see these loops become more uniform or expand to a normal range, signifying an improved ability to open drawers with a smooth, confident pull. Meanwhile, any continued irregularities or abrupt endings in the loops highlight residual motor deficits that may be addressed with additional upper-extremity strengthening or coordination exercises.

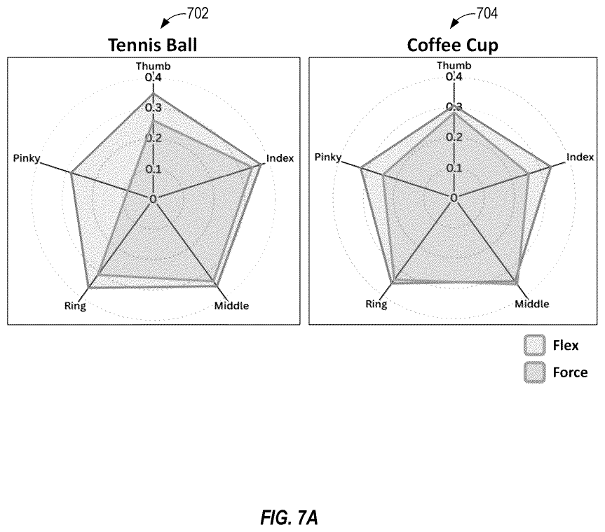

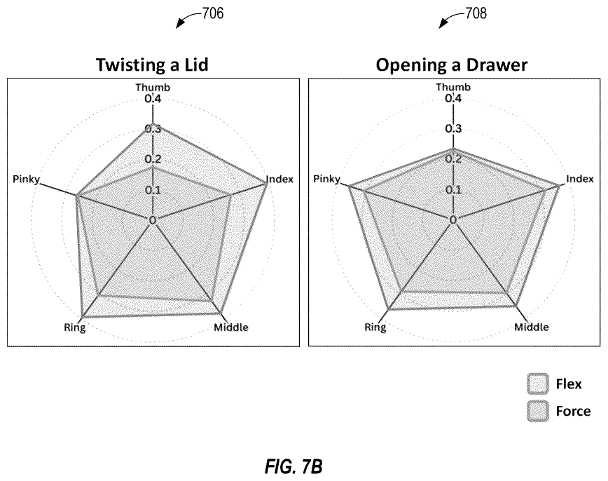

FIGS. 7A and 7B depict exemplary radar-style (or spider) charts derived from principal component analysis (PCA) or related dimensionality-reduction methods, illustrating distinct patterns of flexion and force across the five digits (thumb, index, middle, ring, and pinky) during four different tasks. By combining flex measurements (lightly shaded outline) and force measurements (darker shaded outline) on the same radial axes, these figures offer a comparative view of which fingers are most heavily engaged in each functional activity. In some embodiments, each axis on the pentagon corresponds to a finger, with radial distance representing the normalized amplitude of either flex or force. A user or clinician can thus identify whether a given digit contributes predominantly to grip force or flexion for a particular task, informing targeted rehabilitation strategies. The tasks shown in FIG. 7A are tennis ball PCA chart 702 and coffee cup manipulation PCA chart 704, whereas FIG. 7B illustrates twisting a lid PCA chart 706 and opening a drawer PCA chart 708. By examining the degree of overlap and the shape of each polygon, an individual's synergy or imbalance among the digits can be used to pinpoint weaknesses or guide therapeutic interventions.

Tennis ball PCA chart 702 corresponds to a radar/polar chart (sometimes referred to as a spider chart) that visualizes the relative flex (lightly-shaded polygon) and force (darker-shaded polygon) distributions across the thumb, index, middle, ring, and pinky fingers when a user performs a “tennis ball” task. In certain implementations, the tennis ball exercise entails picking up, holding, or gently squeezing a standard tennis ball—an activity that tests moderate grip force and coordinated flexion. The chart of 702 is typically constructed by first measuring each digit's normalized contribution to flexion and force (e.g., via bend sensors and FSRs) and then projecting these measurements into a low-dimensional PCA space. The chart's radial axes represent the five fingers, while the radial magnitude captures average or peak sensor readings. If the lightly shaded region extends significantly along the ring or pinky axis, it can indicate that these digits are especially involved in gripping the tennis ball. Meanwhile, if the darker shaded area near the thumb or index finger is large, it may imply that these digits provide the majority of force during the grasp. By overlaying the flex and force polygons, clinicians can quickly ascertain whether the user's grip strategy is balanced or heavily reliant on certain digits, potentially revealing deficits in synergy or in the ability to distribute force evenly. Over repeated sessions or among different participants, the shape and size of these polygons can be compared to normative patterns, guiding therapy for individuals with reduced range of motion or compromised finger strength. The tennis ball PCA chart 702 can be used as a high-level diagnostic tool for summarizing how each finger contributes to a typical mid-force grasp in a functional, everyday-like activity.

Coffee cup manipulation PCA chart 704 depicts a radar-style chart for the “coffee cup” task, illustrating PCA-derived flex (lightly-shaded) and force (darker-shaded) profiles across the five digits. In a typical coffee cup exercise, the user grasps a mug by its handle, lifts it up (possibly simulating a drinking motion), and replaces it on a flat surface. This task not only requires coordinated flexion but also places distinct demands on pinch configuration (especially the thumb and index finger) to maintain stability on the cup's handle. The chart in 704 shows, for each digit, how much flexion (lightly-shaded polygon) and force (darker-shaded polygon) are exerted, as normalized to a maximum or mean reference. If the user predominantly employs the thumb and index finger, the pink and purple polygons might bulge outward along those two axes, indicating higher activation there. Conversely, if the ring or middle finger also extends significantly, the polygons could form a more rounded shape, signifying a more balanced approach. Clinically, noticing a large disparity—for instance, the pink polygon is robust for ring and pinky while the purple polygon remains small—might suggest that while the user is comfortable curling those fingers, they are not applying much grip force with them. By systematically recording these coffee cup charts over multiple trials or therapy sessions, therapists can measure improvement in fine motor control, wrist stability, or synergy among the digits. Furthermore, advanced embodiments might correlate the shape of the polygons in the coffee cup manipulation PCA chart 704 to measures of task success (e.g., minimal spillage, efficient picking up/putting down motions), thereby providing direct functional feedback and motivation to the user.

Twisting a lid PCA chart 706 of FIG. 7B corresponds to a radar chart that highlights the force-flex distribution when a user performs a “twisting a lid” task. Commonly, this task simulates opening or closing a jar/bottle lid, requiring a well-coordinated clamp from the thumb and multiple fingers, plus forearm rotation. In some implementations, the user tries to rotate the lid several times, and the sensors track each finger's flexion angles as well as how much pressure (or torque) each digit exerts. By applying PCA to the high-dimensional sensor data, a simplified projection emerges, displayed as two overlapping polygons-lightly-shaded for flex and darker-shaded for force. On the horizontal ring-middle-index axes, for instance, the chart might show that the ring finger exerts a moderate grip force but minimal flex movement, whereas the index finger has a higher flex reading but less force. Such asymmetries can signify compensation strategies or highlight a motor coordination deficit. If the user exhibits a large discrepancy between the lightly-shaded and darker-shaded polygons along the pinky or thumb axis, it could indicate that, while the user bends that digit sufficiently, they fail to apply meaningful torque or grip. Clinically, the twisting a lid PCA chart 706 offers a quick snapshot of whether the user can effectively distribute force among multiple digits or is over-relying on one or two. Over time, a more balanced shape might emerge as therapy improves the individual's rotational dexterity and synergy, lessening the risk that jars remain sealed or that the user experiences hand fatigue from overcompensating with specific fingers.

Opening drawer PCA chart 708 in FIG. 7B depicts a PCA-based spider chart for the “opening a drawer” task, a common assessment for mid-range pulling force and finger extension. Typically, the user must grip a handle or knob, apply an outward pull to slide the drawer open, and subsequently push it back to close. Each finger's sensor data, capturing how far it flexes and how much force it contributes, are aggregated and reduced via PCA, yielding two polygons that reflect flex (lightly-shaded) and force (darker-shaded) patterns. In many drawer-related grips, the ring and pinky fingers may exhibit less force, with the index and middle fingers taking on more pulling load. If the chart's darker-shaded polygon strongly expands toward the index axis, it implies that the finger is important in applying outward force, whereas minimal expansion for the ring axis may indicate lesser engagement there. The lightly shaded shape, on the other hand, might reveal high flex values for the ring finger—indicating it curls significantly, even if it does not deliver much pulling force. Clinically, the shape of the chart in 708 can reveal whether the user's technique is balanced or if they rely excessively on the thumb-index pinch for pulling actions. Repeated measurements across sessions can track improvements in distributed grip strength, thus offering an objective measure of progress in daily living tasks. For instance, a user might gradually learn to incorporate more ring-pinky force, alleviating strain on the index finger over time. Hence, 708 offers an at-a-glance depiction of the synergy between flex and force for a practical, real-world pulling maneuver-helping clinicians pinpoint where motor deficits persist and whether targeted training for particular digits might increase the user's functional independence.

In some aspects, opening drawer PCA chart 708 in FIG. 7B depicts a PCA-based spider chart aggregating data from all participants performing the “opening a drawer” task, which assesses mid-range pulling force and finger extension. The user must grip a handle or knob, apply an outward pull to slide the drawer open, and subsequently push it back to close. The pooled sensor data from all participants, capturing flex and force measurements, are aggregated and reduced via PCA, yielding two polygons that reflect overall flex (lightly-shaded) and force (darker-shaded) patterns across participants. The chart reveals general patterns in finger engagement—for example, if the darker-shaded polygon expands toward the index axis, it indicates this finger's importance in applying outward force across participants. Similarly, if the lightly-shaded shape shows high flex values for certain fingers, this indicates consistent curling patterns even if those digits don't contribute significant force. Hence, 708 provides an at-a-glance depiction of typical finger synergies during this practical pulling maneuver across multiple users.

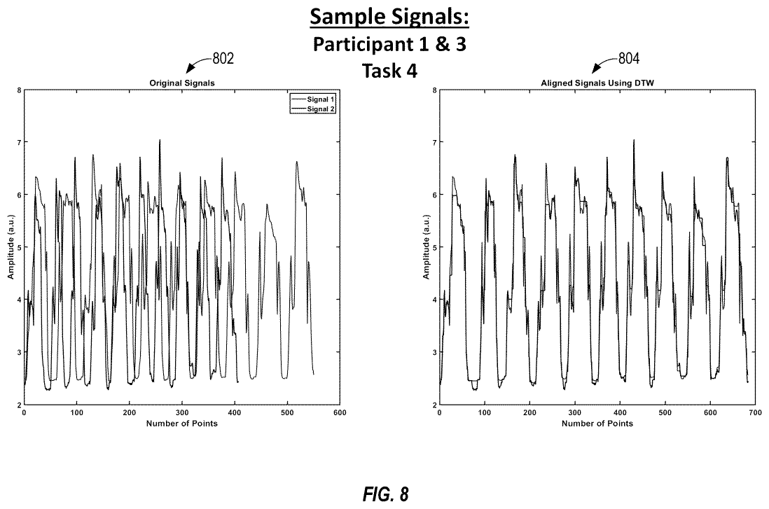

FIG. 8 depicts exemplary time-series data collected from one or more sensors (e.g., sensor 502) representing user hand-movement signals for a particular task (in this instance, “Task 4”). Two subplots are depicted side-by-side: the left subplot 802 shows the original signals, while the right subplot 804 displays the aligned signals after applying a dynamic time warping (DTW) procedure (e.g., from the DTW component 513 discussed previously). By juxtaposing these two plots, it becomes apparent how DTW can compensate for differences in timing, speed, or phase between two distinct signal traces, thereby permitting a more direct comparison of their underlying shapes or amplitude patterns.

Reference character 802 denotes the left-side subplot, which depicts two raw or original signal traces labeled “Signal 1” (dark) and “Signal 2” (light). In various embodiments, these signals may represent sensor 502 data capturing finger flexion angles, applied grip force, or electromyographic (EMG) activity from different participants or different sessions. For example, one might record “Signal 1” from Participant 1 and “Signal 2” from Participant 3 performing an identical motor task; despite the task being the same, natural variations in speed or pacing can cause the two traces to appear misaligned along the time axis. Additionally, movement hesitations, brief rests, or faster transitions by one participant can yield mismatched cycles and peaks. As seen in 802, the amplitude (shown on the vertical axis) reflects some measurement of motor output—such as joint angle or force—while the horizontal axis denotes the “Number of Points,” which can either represent the sampling index or time steps. If a clinician attempts to compare the signals directly in the “original” form, it may be challenging to isolate whether differences arise from legitimate dissimilarities in movement patterns or merely from one participant moving faster or pausing at different intervals. As a result, the unaligned signals in 802 serve as a baseline illustration of how the same functional task can yield visually divergent waveforms, complicating any analysis that relies on cycle-by-cycle matching or amplitude comparisons. Through further processing (e.g., dynamic time warping), these differences can be partially neutralized, enabling the system to identify deeper similarities or discrepancies in the users' motor strategies.

Reference character 804 corresponds to the right-side subplot in FIG. 8, wherein the same two signals (Signal 1 and Signal 2) have been subjected to an alignment procedure, such as dynamic time warping (DTW) implemented by the DTW component 513 of FIG. 5. Following DTW, the signals appear more synchronized in both phase and amplitude, which becomes evident by the near-coincidence of key peaks, troughs, and transitions. In certain implementations, DTW scans through the two waveforms, calculates local distance measures, and determines an optimal path through a cost matrix to “warp” the time axes such that corresponding events (e.g., peak force or maximum finger extension) align more closely. This process effectively stretches or compresses one signal's timeline without altering the essential shape of its amplitude profile, thereby preserving the inherent movement characteristics. Clinically, 804 clarifies whether the two participants (or two separate trials from the same participant) exhibit fundamentally similar movement patterns once speed or timing differences are accounted for. For instance, if the signals remain highly correlated post-DTW, it suggests that both participants execute the underlying movement in a similar manner if one physically moves faster or inserts pauses. Conversely, if large residual discrepancies persist despite alignment, that may indicate truly distinct motor strategies or substantial variability in force/angle usage. Thus, 804 provides a different type of comparison, aiding clinicians or researchers in discerning whether observed variations stem from superficial timing issues or from deeper, clinically relevant deviations in the user's technique.

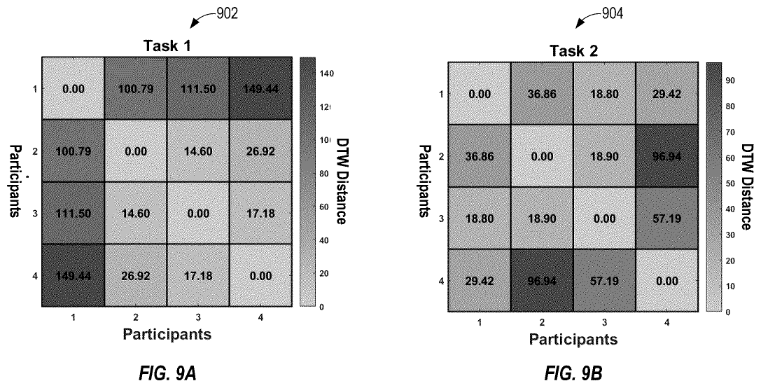

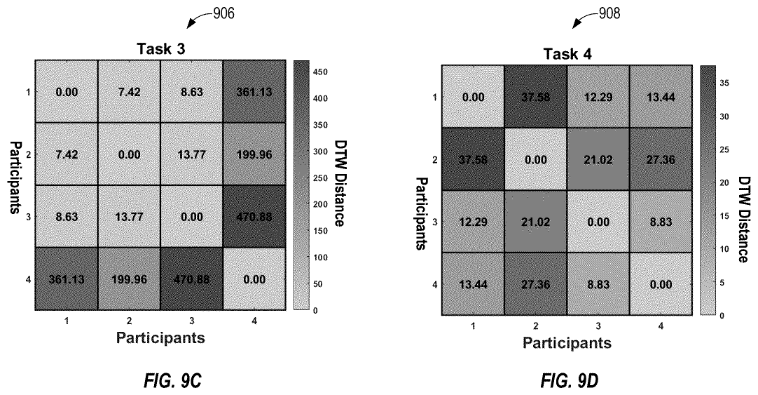

FIGS. 9A-9D illustrate exemplary pairwise distance charts, each derived via a dynamic time warping (DTW) analysis of participants' signals collected during four distinct tasks. In general, a dynamic time-warping representation compares two time-series waveforms and computes a minimal “alignment cost” that indicates how similar or dissimilar those two signals are once speed and phase disparities are accounted for. In FIGS. 9A-9D, each matrix cell corresponds to the DTW distance between two participants, with a color scale signifying numerical distance (ranging from smaller values in a darker color toward higher values in lighter colors). Diagonal cells, representing each participant's distance to themselves, are naturally zero. These charts allow researchers or clinicians to see at a glance how closely any two participants' motions matched when performing the same task-be it opening a drawer, twisting a lid, or another predefined activity. If two participants' squares are a dark color, it implies a relatively large distance, suggesting their signals differ significantly; conversely, a lighter or bluer square indicates more similarity. In a rehabilitation context, such comparisons can reveal whether a patient's pattern approaches that of an unimpaired reference participant or if the patient remains substantially different in the timing and shape of the movement. The following references further detail each chart for Tasks 1 through 4.

Chart 902 designates the pairwise DTW distance matrix for Task 1. In one embodiment, Task 1 might involve picking up a small object or completing a simple hand-closure and release cycle. The rows and columns both represent participants, such that each cell in the grid shows the DTW alignment cost, comparing the row participant's signal to the column participant's signal. A diagonal value of zero appears when a participant is compared to themselves. Off-diagonal cells can vary widely, with some indicating a higher DTW distance, meaning the two participants move in distinct ways for Task 1 (different speeds, incomplete cycles, or hesitations), whereas some cells imply more closely aligned signals. If the top row features notably large distances when comparing Participant 1 to others, that might suggest Participant 1 performed the Task 1 motion quite differently. This matrix thus summarizes relative movement dissimilarities across a study group, spotlighting potential outliers, pairs with near-identical motion, or overall variance within the population. Clinicians can consult chart 902 to see if a patient's Task 1 approach is converging on normative patterns over repeated sessions or remains outlying with a large DTW distance to unimpaired peers.

Chart 904 corresponds to the Task 2 DTW distance matrix, following a similar format as chart 902, but for a different functional activity. In some implementations, Task 2 might require the participant to grip an object with a handle (e.g., a coffee cup) or execute an extension-flexion cycle under different constraints than Task 1. Because each participant's signals may vary in amplitude and tempo from task to task, the DTW distances in chart 904 can differ drastically from those in chart 902, even for the same participants. Squares with large numbers might highlight that two participants used distinct gripping strategies-like one employing a power grip and another a more delicate pinch-while smaller value squares would suggest a common execution pattern. By examining the row for a given participant, a therapist can quickly see whether that individual's approach to Task 2 is consistently dissimilar from everyone else or if they match one or more participants fairly closely. In a rehabilitation context, repeated iteration of Task 2 can help measure progress, with decreasing DTW distances signifying an evolving technique that more closely aligns with the normative or group average. Over time, analyzing how chart 904 changes alongside other tasks can expose differential improvements: perhaps a patient's Task 2 performance normalizes faster than Task 1,prompting therapy adjustments to prioritize tasks that remain more challenging.

Chart 906 denotes the DTW distance matrix for Task 3, potentially a moderate-to-complex movement such as twisting a jar lid or turning a doorknob. This chart may display a broader range of distances, reflecting the possibility that some participants adopt profoundly different rotational or torque-driven strategies. If squares exceed certain numeric thresholds (e.g., 300-400), that suggests major misalignment in waveforms even after dynamic time warping, perhaps because one participant's movement is scattered by multiple short attempts, whereas another performs a smooth, continuous twist. Alternatively, squares tinted near the lower end of the scale indicate participants with nearly identical patterns once pacing differences are removed. Clinicians may review chart 906 to identify which participants might share a common technique in rotating the wrist and applying finger torque, or to isolate a single participant who remains an outlier with highly sporadic signals. As a result, this chart can be an indicator of whether a user struggling with rotational tasks is progressing toward more typical motor synergy or if specialized intervention for twisting motions is warranted. By comparing the squares from early sessions to later ones, improvements appear, such as a shift from large-value squares toward smaller-value squares.

Chart 908 identifies the final matrix in FIG. 9D, covering Task 4—another discrete functional activity (e.g., opening a drawer, performing repeated pinch grips, or any designated motion requiring cyclical force). In contrast to the large numeric range in chart 906, the color bar here might max out near 35, indicating that overall, the group's signals for Task 4 are more similar or require fewer sample points to warp into alignment. A large value square in chart 908 still means a substantially different shape or timing but at a smaller absolute scale. Reading across the matrix, a clinician or researcher can glean whether participants 1 and 2 differ from 3 and 4 in their approach or if a single participant diverges from the rest. Because Task 4 might emphasize linear pulling force or extension rather than rotation, participants who excel in simpler, more linear tasks could exhibit low pairwise distances with others who adopt a similarly direct approach. Conversely, those with less stable force application or inconsistent grip might yield moderate to high distances. This matrix thus completes the set of dynamic time-warping comparisons for all tasks, depicting how each participant's technique stands relative to their peers under various motor demands.

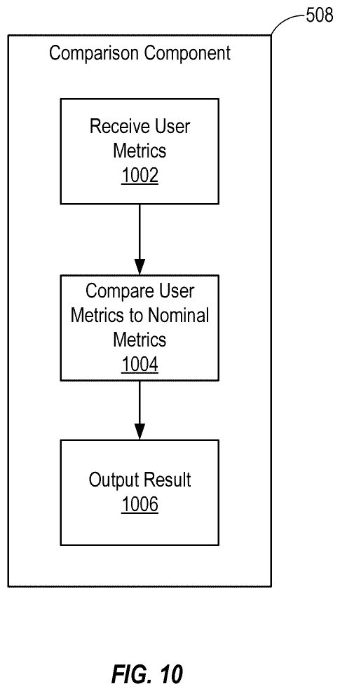

FIG. 10 illustrates an exemplary comparison component 508 that receives, evaluates, and provides results based on user-specific data (e.g., sensor-derived measurements or clinically derived performance indices). In certain embodiments, the comparison component is implemented within a processing system (such as a machine-learning engine or a simpler heuristic unit) that aims to gauge the user's progress or current status relative to predefined or dynamically generated baselines. By automatically matching user data against known references—sometimes referred to herein as “reference metrics”—the system can output a diagnostic or feedback score indicating whether the user's performance is improving, stable, or regressing in a given task.

The comparison component 508 may be encapsulated in software, firmware, or a combination of hardware and algorithmic logic. Typically, the comparison component 508 interfaces with upstream modules (e.g., metric processor 504 of FIG. 5) to collect consolidated data from sensors, principal component analysis, or dynamic time-warping modules. The comparison component's 508 functionality extends beyond mere numeric matching: it can normalize, filter, or weigh incoming measurements before conducting comparisons. In a rehabilitation setting, the comparison component 508 can interpret the user's force application patterns, flexion angles, or aggregated synergy scores in the context of established normative benchmarks. The comparison component 508 may be scalable and configurable, allowing a clinician to define multiple reference profiles such as typical non-impaired adults, individuals with mild impairments, or the user's own baseline from an earlier session. These profiles can be used to generate comparative metrics that reflect how closely the user's current performance aligns with any chosen reference. By encapsulating the “receive, compare, and output” steps, the comparison component 508 generates decision-support information, potentially triggering an alert or generating instructions for further intervention if the user's data deviates from expected thresholds.

At 1002, a step or subcomponent receives user metrics, which may encompass raw sensor readings, post-processed signals, or aggregated scores derived from earlier phases of the system pipeline. These user metrics could be anything from time-series waveforms (e.g., flex sensor outputs over a task duration) to higher-level indexes (e.g., principal component synergy factors, dynamic time warping scores, or standard clinical tests like Fugl-Meyer sub-scores). Depending on the configuration, 1002 might poll a data buffer at scheduled intervals, subscribe to real-time streaming from the metric processor, or retrieve historical records from a local or cloud-based repository. In some embodiments, 1002 also performs validation checks to ensure that incoming metrics conform to expected ranges (e.g., no negative angles, no excessively large force values) or remain logically consistent with the user's known capabilities. By consolidating relevant data into an internal structure, 1002 sets the stage for subsequent evaluations. This ensures that the metrics are in a standardized format before proceeding to the comparison step, making the entire process more robust and less prone to erroneous conclusions. Furthermore, by tracking timestamps or session IDs, 1002 can align user metrics with specific tasks so that the system only compares “like with like”—for instance, matching a user's “Tennis Ball” performance metrics to corresponding reference data for that same functional task.

At 1004, the system may compare user metrics to reference metrics, where “reference” can include normative data, non-impaired control averages, previously recorded user baselines, or clinically established performance thresholds. The nature of the comparison may vary: in one example, the system calculates a distance measure (e.g., Euclidean distance or a time-warped correlation) between the user's synergy vector and a standard vector. In another instance, 1004 checks if the user's grip strength (captured in Newton equivalents) meets a threshold typical of non-impaired adults. This step can involve statistical or machine-learning algorithms, such as computing a z-score, outlier detection, or classification using a pre-trained model. The system might also factor in demographic variables like age or handedness so that the user's data are only compared to appropriate subgroups within the reference dataset. Through this multi-faceted evaluation, the comparison at 1004 produces an intermediate metric (or set of metrics) indicating how closely (or poorly) the user's results align with target expectations. When integrated into a larger rehabilitation framework, these comparisons can guide therapists or clinicians in tailoring exercises or automatically adapting task difficulty if the user is consistently surpassing or failing to meet expected performance bands.

At 1006, the result may be output. For example, the comparison, which could be a numeric score, a descriptive label (e.g., “improved,” “within normal limits,” “needs more practice”), or a visual indicator (such as a color-coded progress bar) may be provided to a user, clinician, or therapist. In some configurations, 1006 can generate real-time feedback to display on a user interface, informing the patient or therapist immediately about how well the user performed relative to benchmark data. Alternatively, the output might be stored in a database, sent as a summary report to a remote clinician, or employed to auto-adjust the difficulty level in a therapy session. The method of output can range from textual feedback (“Your performance is 85% of the healthy norm”) to more sophisticated dashboards plotting multi-session trends. Additionally, if the system identifies severe deviations from expected progress, step 1006 might trigger alerts or suggest a re-evaluation of the user's therapy goals. By centralizing the “output result” step, step 1006 underscores the system's role in bridging raw data analysis with meaningful, actionable outcomes for both clinical and research environments.

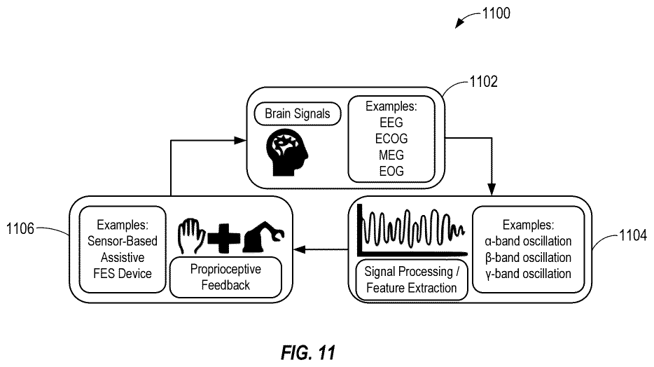

FIG. 11 provides an exemplary overview of a multi-stage system 1100 wherein brain signals are collected and processed to facilitate real-time feedback or assistance for a user's motor function. In certain implementations, the system 1100 gathers neural data using one or more modalities (e.g., EEG, ECOG, MEG, EOG), extracts meaningful features from distinct frequency bands (e.g., alpha, beta, gamma), and subsequently supplies proprioceptive or mechanized feedback. This bidirectional approach leverages neural signals as a trigger or indicator of the user's intended movement and then delivers tangible feedback to reinforce or guide correct motor patterns, potentially aiding rehabilitation or general motor training. The following descriptions provide further detail for each reference character shown.

In some aspects, the brain signals acquired from the user at 1102 may be captured by various sensing techniques such as electroencephalography (EEG), electrocorticography (ECoG), magnetoencephalography (MEG), or electrooculography (EOG). In certain aspects, a user dons a noninvasive EEG cap or an MEG helmet, whereas more advanced setups may incorporate invasive electrodes placed atop or within the cortical surface (e.g., ECoG). The signals obtained typically contain oscillations in multiple frequency ranges, each potentially indicative of different aspects of the user's motor intent, cognitive load, or visual engagement. For example, alpha-band rhythms may correlate with resting states or motor imagery, whereas beta-band patterns might reflect active motor processing or sensorimotor control. Additionally, EOG signals can identify eye movements or blinks that could otherwise contaminate the recordings or be exploited as additional control inputs. By acquiring and digitizing these brain signals, reference character 1102 forms the input layer of the overall system, effectively capturing the user's neural activity in real-time. In a rehabilitation context, these signals can inform a higher-level brain-computer interface (BCI) or control scheme, allowing the system to identify when the user is planning a particular movement (e.g., grasping) or to detect if the user is imagining a specific hand action despite having limited volitional control. In some instances, the system calibrates brain signal acquisition by instructing the user to perform or imagine repeated motions while the device records baseline patterns for each frequency band, thus refining the subsequent signal-processing steps.

In some aspects, the acquired data may be processed at 1104, which operates on the raw neural data derived from 1102. Through computational algorithms such as band-pass filtering, Fourier transforms, wavelet decomposition, or machine-learning classifiers, the system isolates key features embedded in alpha-, beta-, and gamma-band oscillations. In one illustrative embodiment, alpha-band activity might be filtered in the 8-12 Hz range, beta in 13-30 Hz, and gamma in 30-100 Hz or higher, though exact cutoffs can vary. These frequency components often correlate with distinct neural processes: alpha rhythms may signal relaxation or motor imagery blocking, beta rhythms can link to active motor control or motor intentions, and gamma oscillations are frequently associated with heightened attentional or sensory processing demands. Once extracted, the features may be used to classify user intent in opening or closing the hand or to compute a continuous control signal reflecting motor effort or engagement. The algorithms may further employ artifact removal (e.g., removing eye-blink signals) or dimensionality reduction techniques (e.g., principal component analysis) to improve the signal-to-noise ratio. Signal processing 1104 may output an interpretable representation (such as a real-time event trigger or a continuous “movement readiness” metric), enabling robust detection of the user's volitional or imagined movements. This extracted information then feeds forward into the device's feedback loop, guiding the assistive or rehabilitative interventions that follow.

Reference character 1106 denotes the system's proprioceptive feedback stage, exemplified by a sensor-based assistive device or a functional electrical stimulation (FES) apparatus. In some aspects, once the signal processing 1104 confirms the user's motor intent (e.g., to grip an object), the system can deliver corresponding feedback directly to the user's limb. In some embodiments, FES electrodes placed on the user's forearm stimulate appropriate muscles to reinforce the intended movement, thereby bridging the gap between brain intent and actual motion. Alternatively, a wearable exoskeleton or robotic brace might physically guide the user's hand through the desired grasping motion. A sensor array integrated into that device can measure force or displacement, ensuring that the user experiences a realistic sense of proprioception (i.e., feeling how the limb moves and how much force is being applied). This feedback loop helps retrain neural pathways, as the user's brain signals 1102 lead to a physical action, which in turn is sensed by the body, reinforcing synaptic connections critical to motor learning or rehabilitation. Over multiple trials, reference character 1106 can adapt the level of assistance or stimulation, slowly phasing out external support as the user's voluntary control recovers. This mechanism is especially valuable for stroke survivors or individuals with partial paralysis, allowing them to harness brain activity to drive real, tangible limb movements and thereby reestablish functional movement patterns.

Moreover, in certain implementations, 1106 may include a display or user-facing interface that illustrates the individual's neural intent side-by-side with the actual motion as recorded by sensor gloves or other instrumentation. For instance, the interface might show a visual gauge of “predicted grip force” from the brain signal next to a real-time readout of the force truly exerted on a test object. Where these two measurements differ—i.e., the person “intends” more force than they actually produce-clinicians can identify possible neuromuscular deficits or spasticity and adjust therapy accordingly. Consequently, rehabilitation protocols, diagnostic decisions, or broader clinical assessments can account for both intent and outcome, tailoring each patient's plan to address discrepancies between the neural command and executed movement. Over time, repeated feedback cycles can train the patient to better align brain signals with actual muscle activation, improving motor control in daily tasks.

### Example Operations for Generating a Performance Measure

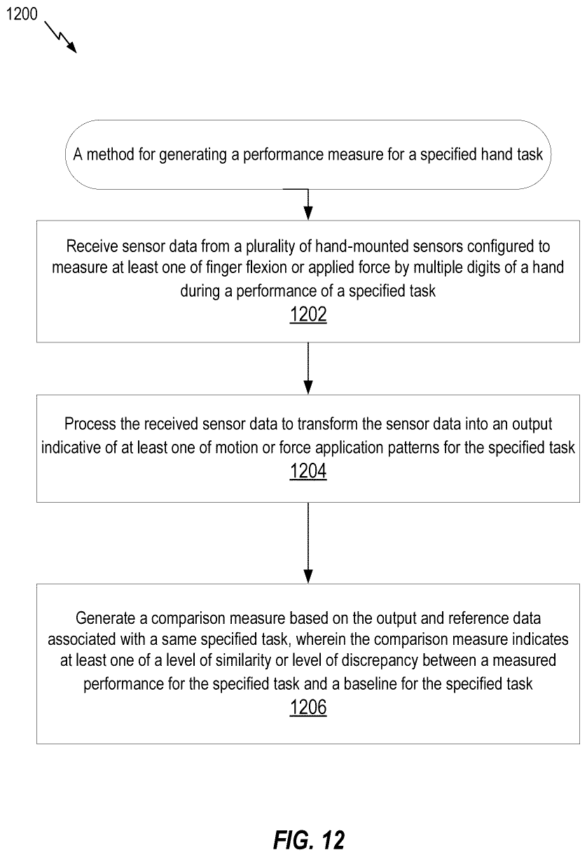

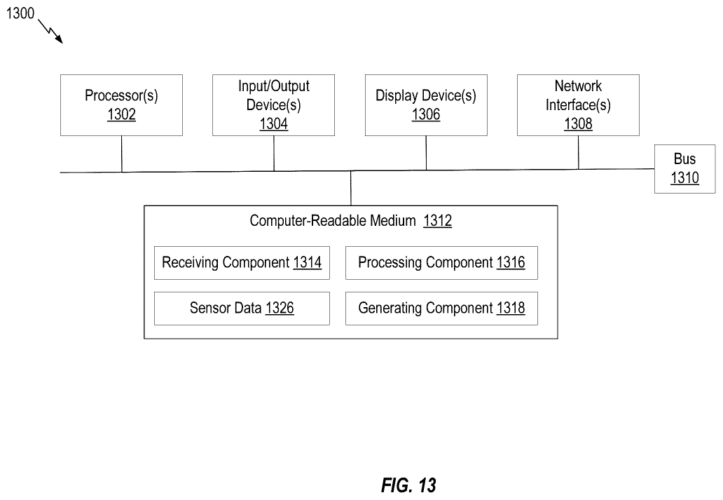

FIG. 12 depicts an example method 1200 for generating a performance measure for a specified hand task. In one aspect, method 1200, or any aspect related to it, may be performed by an apparatus, such as processing system 1300 of FIG. 13, which includes various components operable, configured, or adapted to perform method 1200. In one aspect, method 1200 can be implemented by the processing system 114 or processing component 116 of FIG. 1.

Method 1200 starts at block 1202 with receiving sensor data from a plurality of hand-mounted sensors configured to measure at least one of finger flexion or applied force by multiple digits of a hand during a performance of a specified task. For example, sensor data may be obtained from one or more flex sensors 108 and/or one or more FSR sensors 112 of FIG. 1 during the performance of a task, such as an extension tasks, contraction task, tennis ball task, coffee cup task, twisting a lid task, or opening a drawer task as previously described.

Method 1200 continues to block 1204 with processing the received sensor data to transform the sensor data into an output indicative of at least one of motion or force application patterns for the specified task. In one aspect, one or more of the processing system 114 or processing component 116 of FIG. 1 may transform the sensor data into an output, such as a return map. As another example, the metric processor 504 may provide an output representation 506, as indicated in FIG. 5.

Method 1200 continues to block 1206 with generating a comparison measure based on the output and reference data associated with a same specified task, wherein the comparison measure indicates at least one of a level of similarity or level of discrepancy between a measured performance for the specified task and a baseline for the specified task. In one aspect, one or more of the processing system 114 or processing component 116 of FIG. 1 may provide a comparison measure. In one aspect, the metric processor 504 may provide a comparison component 508, as indicated in FIG. 5.

Note that FIG. 12 is just one example of a method, and other methods, including fewer, additional, or alternative steps, are possible consistent with this disclosure.

### Example Processing System for Generating a Performance Measure

FIG. 13 depicts aspects of an example processing system 1300.

Processing system 1300 is generally an example of an electronic device configured to execute computer-executable instructions, such as those derived from compiled computer code, including without limitation personal computers, tablet computers, servers, smartphones, smart devices, wearable devices, augmented and/or virtual reality devices, and others.

In the depicted example, processing system 1300 includes one or more processor(s) 1302, one or more input/output devices 1304, one or more display devices 1306, one or more network interfaces 1308 through which processing system 1300 is connected to one or more networks (e.g., a local network, an intranet, the Internet, or any other group of processing systems communicatively connected to each other), and computer-readable medium 1312. In the depicted example, the aforementioned components are coupled by a bus 1310, which may generally be configured for data exchange amongst the components. Bus 1310 may be representative of multiple buses, while only one is depicted for simplicity.

Processor(s) 1302 are generally configured to retrieve and execute instructions stored in one or more memories, including local memories like computer-readable medium 1312, as well as remote memories and data stores. Similarly, processor(s) 1302 are configured to store application data residing in local memories like the computer-readable medium 1312, as well as remote memories and data stores. More generally, bus 1310 is configured to transmit programming instructions and application data among the processor(s) 1302, display device(s) 1306, network interface(s) 1308, and/or computer-readable medium 1312. In certain embodiments, processor(s) 1302 are representative of one or more central processing units (CPUs), graphics processing units (GPUs), tensor processing units (TPUs), accelerators, and other processing devices.

Input/output device(s) 1304 may include any device, mechanism, system, interactive display, and/or various other hardware and software components for communicating information between processing system 1300 and a user of processing system 1300. For example, input/output device(s) 1304 may include input hardware, such as a keyboard, touch screen, button, microphone, speaker, and/or other device for receiving inputs from the user and sending outputs to the user.

Display device(s) 1306 may generally include any sort of device configured to display data, information, graphics, user interface elements, and the like to a user. For example, display device(s) 1306 may include internal and external displays, such as an internal display of a tablet computer or an external display for a server computer or a projector. Display device(s) 1306 may further include displays for devices, such as augmented, virtual, and/or extended reality devices. In various embodiments, display device(s) 1306 may be configured to display a graphical user interface.

Network interface(s) 1308 provides processing system 1300 with access to external networks and, thereby, to external processing systems. Network interface(s) 1308 can generally be any hardware and/or software capable of transmitting and/or receiving data via a wired or wireless network connection. Accordingly, network interface(s) 1308 can include a communication transceiver for sending and/or receiving any wired and/or wireless communication.

Computer-readable medium 1312 may be a volatile memory, such as a random access memory (RAM), or a nonvolatile memory, such as nonvolatile random access memory (NVRAM), or the like. In certain aspects, the computer-readable medium/memory 1312 is configured to store instructions (e.g., computer-executable code) that when executed by the one or more processor(s) 1302, cause the one or more processor(s) 1302 to perform the method 1200 described with respect to FIG. 12, or any aspect related to it, including any additional steps or sub-steps described in relation to FIG. 12.

In the depicted example, computer-readable medium/memory 1312 stores code (e.g., executable instructions) for a receiving component 1314, code for a processing component 1316, and code for a generating component 1318. Processing of the code for the receiving component 1314, code for the processing component 1316, and/or code for the generating component may enable and cause the processing system 1300 to perform the method 1200 described with respect to FIG. 12 or any aspect related to it. The one or more processor(s) 1302 may include circuitry configured to implement (e.g., execute) the code stored in the computer-readable medium/memory 1312.

In some aspects, the receiving component 1314 may receive sensor data, such as sensor data obtained from one or more flex sensors 108 and/or one or more FSR sensors 112 of FIG. 1 during the performance of a task, such as an extension tasks, contraction task, tennis ball task, coffee cup task, twisting a lid task, or opening a drawer task as previously described. In some aspects, the sensor data may be stored as sensor data 1326.

In some aspects, the processing component 1316 may process the received sensor data to transform the sensor data into an output indicative of at least one of motion or force application patterns for the specified task. In one aspect, the processing component 1316 may correspond to one or more of the processing system 114 or processing component 116 of FIG. 1 and may transform the sensor data into an output, such as a return map. As another example, the processing component 1316 may correspond to the metric processor 504 of FIG. 5.

In some aspects, the generating component 1318 may generate a comparison measure based on the output and reference data associated with a same specified task, wherein the comparison measure indicates at least one of a level of similarity or level of discrepancy between a measured performance for the specified task and a baseline for the specified task. In one aspect, the generating component 1318 may correspond to one or more of the processing system 114 or processing component 116 of FIG. 1 and may provide a comparison measure. In one aspect, the generating component 1318 may correspond to the metric processor 504 of FIG. 5.

### Additional Considerations

The preceding description is provided to enable any person skilled in the art to practice the various aspects described herein. The examples discussed herein are not limiting of the scope, applicability, or aspects set forth in the claims. Various modifications to these aspects will be readily apparent to those skilled in the art, and the general principles defined herein may be applied to other aspects. For example, changes may be made in the function and arrangement of elements discussed without departing from the scope of the disclosure. Various examples may omit, substitute, or add various procedures or components as appropriate. For instance, the methods described may be performed in an order different from those described, and various actions may be added, omitted, or combined. Also, features described with respect to some examples may be combined in some other examples. For example, an apparatus may be implemented, or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

The various illustrative logical blocks, modules, and circuits described in connection with the present disclosure may be implemented or performed with a general-purpose processor, a digital signal processor (DSP), an ASIC, a field programmable gate array (FPGA), or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, a system on a chip (SoC), or any other such configuration.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database, or another data structure), ascertaining, and the like. Also, “determining” may include receiving (e.g., receiving information), accessing (e.g., accessing data in a memory), and the like. Also, “determining” may include resolving, selecting, choosing, establishing, and the like.

As used herein, “coupled to” and “coupled with” generally encompass direct coupling and indirect coupling (e.g., including intermediary coupled aspects) unless stated otherwise. For example, stating that a processor is coupled to memory allows for a direct coupling or a coupling via an intermediary aspect, such as a bus.

The methods disclosed herein comprise one or more actions to achieve the methods. The method actions may be interchanged with one another without departing from the scope of the claims. In other words, unless a specific order of actions is specified, the order and/or use of specific actions may be modified without departing from the scope of the claims. Further, the various operations of methods described above may be performed by any suitable means capable of performing the corresponding functions. The means may include various hardware and/or software component(s) and/or module(s), including, but not limited to, a circuit, an application-specific integrated circuit (ASIC), or a processor.

The following claims are not intended to be limited to the aspects shown herein but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public, regardless of whether such disclosure is explicitly recited in the claims.

What is claimed is:

1. A system for evaluating a hand function for a specified task, the system comprising:
a plurality of hand-mounted sensors configured to measure at least one of finger flexion or applied force by multiple digits of a hand during a performance of a specified task;
one or more memories comprising processor-executable instructions; and
one or more processors configured to execute the processor-executable instructions and cause the system to:
receive sensor data from the plurality of hand-mounted sensors;
process the received sensor data to transform the sensor data into an output indicative of at least one of motion or force application patterns for the specified task; and
generate a comparison measure based on the output and reference data associated with a same specified task, wherein the comparison measure indicates at least one of a level of similarity or level of discrepancy between a measured performance for the specified task and a baseline for the specified task.

2. The system of claim 1, wherein the instructions, when executed by the one or more processors, further cause the system to pre-process the sensor data to at least one of remove noise or reduce a sampling rate prior to transforming the sensor data into the output.

3. The system of claim 1, wherein the instructions, when executed by the one or more processors, further cause the system to apply one or more transformations comprising:
dimensionality reduction;
time-alignment; or
delay embedding.

4. The system of claim 3, wherein the processed output comprises at least one of:
a return map of a time-lagged plot of hand movements,
a dynamic time-warping alignment including matched phases between different users, or
a principal component analysis (PCA) projection depicting reduced-dimensionality patterns of motion and/or force profiles.

5. The system of claim 1, wherein the instructions, when executed by the one or more processors, further cause the system to adjust at least one of a clinical or rehabilitative plan based on the comparison measure.

6. The system of claim 1, wherein the instructions, when executed by the one or more processors, further cause the system to perform a calibration process that captures baseline hand movements under controlled conditions and establish one or more reference parameters for subsequent sensor data interpretation.

7. The system of claim 1, wherein the instructions, when executed by the one or more processors, further cause the system to incorporate at least one of electroencephalography (EEG), electrocorticography (ECoG), magnetoencephalography (MEG), or electrooculography (EOG) signals and refine the comparison measure based on user intent.

8. The system of claim 1, wherein the one or more processors are configured to store the comparison measure and associated reference data in a data repository.

9. The system of claim 1, wherein the plurality of hand-mounted sensors comprises at least three sensor elements configured to obtain sensor data for at least three digits during the specified task.

10. The system of claim 1, wherein the reference data is based on a plurality of non-impaired control users completing a same specified task, enabling the comparison measure to quantify the measured performance similarity to or deviation from normative hand function profiles.

11. A method for generating a performance measure for a specified hand task, the method comprising:
receiving, by at least one processor, sensor data from a plurality of hand-mounted sensors configured to measure at least one of finger flexion or applied force by multiple digits of a hand during a performance of a specified task;
processing the received sensor data to transform the sensor data into an output indicative of at least one of motion or force application patterns for the specified task; and
generating a comparison measure based on the output and reference data associated with a same specified task, wherein the comparison measure indicates at least one of a level of similarity or level of discrepancy between a measured performance for the specified task and a baseline for the specified task.

12. The method of claim 11, wherein processing the received sensor data includes pre-processing the sensor data to at least one of remove noise or reduce a sampling rate prior to generating the output indicative of the motion or force application patterns.

13. The method of claim 12, wherein processing the received sensor data includes further applying one or more transformations comprising:
dimensionality reduction;
time-alignment; and
delay embedding.

14. The method of claim 13, wherein the output comprises at least one of:
a return map of a time-lagged plot of hand movements;
a dynamic time-warping alignment including matched phases between different trials or different users; or
a principal component analysis (PCA) projection depicting reduced-dimensionality patterns of motion or force profiles.

15. The method of claim 14, wherein the return map is generated by mapping a time-shifted version of the motion or force signal against an unshifted version and obtaining at least one of a cyclical or repetitive indication in movement data.

16. The method of claim 11, further comprising adjusting at least one of a clinical or rehabilitative plan based on the generated comparison measure.

17. The method of claim 11, further comprising performing a calibration process including capturing baseline hand movements under controlled conditions to establish one or more reference parameters for subsequent sensor data interpretation.

18. The method of claim 11, further comprising refining the comparison measure based on at least one of an electroencephalography (EEG) signal, electrocorticography (ECoG) signal, magnetoencephalography (MEG) signal, or electrooculography (EOG) signal.

19. The method of claim 11, wherein receiving sensor data from the plurality of hand-mounted sensors includes recording sensor data during at least one specified task including at least one of: grasping a ball, manipulating a cup, twisting a lid, or opening a drawer.

20. A computer program product embodied on a computer-readable medium comprising program code for performing a method, the method comprising:
receiving, by at least one processor, sensor data from a plurality of hand-mounted sensors configured to measure at least one of finger flexion or applied force by multiple digits of a hand during a performance of a specified task;
processing the received sensor data to transform the sensor data into an output indicative of at least one of motion or force application patterns for the specified task; and
generating a comparison measure based on the output and reference data associated with a same specified task, wherein the comparison measure indicates at least one of a level of similarity or level of discrepancy between a measured performance for the specified task and a baseline for the specified task.
